SEQUENCE LISTING (46 sequences)

SEQ ID NO: 1 - protein, 33 aa, Artificial Sequence
  Synthethic peptide
  MISC_FEATURE (3)..(3): X is (R)-alpha-methyl,alpha-octenylglycine
  MISC_FEATURE (10)..(10): X is (S)-alpha-methyl,alpha-pentenylglycine
EKXRDLYSKXTAAMSTYTGIFTDQVLSVLKGEE

SEQ ID NO: 2 - protein, 33 aa, Artificial Sequence
  Synthetic peptide
  MISC_FEATURE (14)..(14): X is (S)-alpha-methyl,alpha-pentenylglycine
  MISC_FEATURE (18)..(18): X is (S)-alpha-methyl,alpha-pentenylglycine
EKLRDLYSKSTAAXSTYXGIFTDQVLSVLKGEE

SEQ ID NO: 3 - protein, 33 aa, Artificial Sequence
  Synthetic peptide
  MISC_FEATURE (11)..(11): X is (R)-alpha-methyl,alpha-octenylglycine
  MISC_FEATURE (18)..(18): X is (S)-alpha-methyl,alpha-pentenylglycine
EKLRDLYSKSXAAMSTYXGIFTDQVLSVLKGEE

SEQ ID NO: 4 - protein, 33 aa, Artificial Sequence
  Synthetic peptide
  MISC_FEATURE (15)..(15): X is (R)-alpha-methyl,alpha-octenylglycine
  MISC_FEATURE (22)..(22): X is (S)-alpha-methyl,alpha-pentenylglycine
EKLRDLYSKSTAAMXTYTGIFXDQVLSVLKGEE

SEQ ID NO: 5 - protein, 24 aa, Artificial Sequence
  Synthetic peptide
  MISC_FEATURE (6)..(6): X is (R)-alpha-methyl,alpha-octenylglycine
  MISC_FEATURE (13)..(13): X is S)-alpha-methyl,alpha-pentenylglycine
STAAMXTYTGIFXDQVLSVLKGEE

SEQ ID NO: 6 - protein, 34 aa, Artificial Sequence
  Synthetic peptide
  MISC_FEATURE (3)..(3): X is (R)-alpha-methyl,alpha-octenylglycine
  MISC_FEATURE (10)..(10): X is (S)-alpha-methyl,alpha-pentenylglycine
EKXRDLYSKXTAPAMSTYTGIFTDQVLSVLKGEE

SEQ ID NO: 7 - protein, 21 aa, Artificial Sequence
  Synthetic peptide
  MISC_FEATURE (3)..(3): X is (R)-alpha-methyl,alpha-octenylglycine
  MISC_FEATURE (10)..(10): X is (S)-alpha-methyl,alpha-pentenylglycine
AMXTYTGIFXDQVLSVLKGEE

SEQ ID NO: 8 - protein, 19 aa, Artificial Sequence
  Synthetic peptide
  MISC_FEATURE (1)..(1): X is (R)-alpha-methyl,alpha-octenylglycine
  MISC_FEATURE (8)..(8): X is (S)-alpha-methyl,alpha-pentenylglycine
XTYTGIFXDQVLSVLKGEE

SEQ ID NO: 9 - protein, 29 aa, Artificial Sequence
  Synthetic peptide
  MISC_FEATURE (6)..(6): X is (R)-alpha-methyl,alpha-octenylglycine
  MISC_FEATURE (13)..(13): X is (S)-alpha-methyl,alpha-pentenylglycine
  MOD_RES (25)..(25): AMIDATION
STAAMXTYTGIFXTDQVLSVLKGEEAMID

SEQ ID NO: 10 - protein, 25 aa, Artificial Sequence
  Synthetic peptide
  MISC_FEATURE (6)..(6): X is (R)-alpha-methyl,alpha-octenylglycine
  MISC_FEATURE (13)..(13): X is (S)-alpha-methyl,alpha-pentenylglycine
  MISC_FEATURE (24)..(24): D-Glu
  MISC_FEATURE (25)..(25): D-Glu
STAAMXTYTGIFXTDQVLSVLKGEE

SEQ ID NO: 11 - protein, 24 aa, Artificial Sequence
  Synthetic peptide
  MISC_FEATURE (5)..(5): X is (R)-alpha-methyl,alpha-octenylglycine
  MISC_FEATURE (12)..(12): X is (S)-alpha-methyl,alpha-pentenylglycine
STAAXSTYTGIXTDQVLSVLKGEE

SEQ ID NO: 12 - protein, 34 aa, Artificial Sequence
  Synthetic peptide
EKLRDLYSKSTAPAMSTYTGIFTDQVLSVLKGEE

SEQ ID NO: 13 - protein, 33 aa, Artificial Sequence
  Synthetic peptide
EKLRDLYSKSTAAMSTYTGIFTDQVLSVLKGEE

SEQ ID NO: 14 - protein, 21 aa, Artificial Sequence
  Synthetic peptide
AMSTYTGIFTDQVLSVLKGEE

SEQ ID NO: 15 - protein, 40 aa, Artificial Sequence
  Synthetic peptide
DYLKEVFEKLRDLYEKFTPAVSTYTGIFTDQVLSVLKGEE

SEQ ID NO: 16 - protein, 21 aa, Artificial Sequence
  Synthetic peptide
  MISC_FEATURE (3)..(3): X is (R)-alpha-methyl,alpha-octenylglycine
  MISC_FEATURE (10)..(10): X is bis-pentenylglycine
  MISC_FEATURE (17)..(17): X is (S)-alpha-methyl,alpha-octenylglycine
AMXTYTGIFXDQVLSVXKGEE

SEQ ID NO: 17 - protein, 21 aa, Artificial Sequence
  Synthetic peptide
  MOD_RES (2)..(2): Nle
  MISC_FEATURE (3)..(3)
  MISC_FEATURE (10)..(10)
  MISC_FEATURE (17)..(17)
  MISC_FEATURE (20)..(20): D-Glu
  MISC_FEATURE (21)..(21): D-Glu
AXXTYTGIFXDQVLSVSKGEE

SEQ ID NO: 18 - protein, 21 aa, Artificial Sequence
  Synthetic peptide
  MOD_RES (2)..(2): Nle
  MISC_FEATURE (3)..(3): X is (R)-alpha-methyl,alpha-octenylglycine
  MISC_FEATURE (10)..(10): X is bis-pentenylglycine
  MISC_FEATURE (17)..(17): X is (S)-alpha-methyl,alpha-octenylglycine
  MOD_RES (19)..(19): MeGly
  MISC_FEATURE (20)..(20): D-Glu
  MISC_FEATURE (21)..(21): D-Glu
AXXTYTGIFXDQVLSVXKXEE

SEQ ID NO: 19 - protein, 21 aa, Artificial Sequence
  Synthetic peptide
  MOD_RES (2)..(2): Nle
  MISC_FEATURE (3)..(3): X is (R)-alpha-methyl,alpha-octenylglycine
  MISC_FEATURE (10)..(10): X is bis-pentenylglycine
  MISC_FEATURE (14)..(14): X is (S)-alpha-methyl,alpha-pentenylglycine
  MISC_FEATURE (20)..(20): D-Glu
  MISC_FEATURE (21)..(21): D-Glu
AXXTYTGIFXDQVXSVLKGEE

SEQ ID NO: 20 - protein, 21 aa, Artificial Sequence
  Synthetic peptide
  MOD_RES (2)..(2): Nle
  MISC_FEATURE (3)..(3): X is (R)-alpha-methyl,alpha-octenylglycine
  MISC_FEATURE (10)..(10): X is bis-pentenylglycine
  MISC_FEATURE (14)..(14): X is (S)-alpha-methyl,alpha-pentenylglycine
  MISC_FEATURE (18)..(18): X is alpha-methyl-Lysine
  MISC_FEATURE (20)..(20): D-Glu
  MISC_FEATURE (21)..(21): D-Glu
AXXTYTGIFXDQVXSVLXGEE

SEQ ID NO: 21 - protein, 21 aa, Artificial Sequence
  Synthetic peptide
  MISC_FEATURE (3)..(3): X is (R)-alpha-methyl,alpha-octenylglycine
  MISC_FEATURE (10)..(10): X is (S)-alpha-methyl,alpha-pentenylglycine
  MOD_RES (21)..(21): PYRROLIDONE CARBOXYLIC ACID
AMXTYTGIFXDQVLSVLKGEX

SEQ ID NO: 22 - protein, 21 aa, Artificial Sequence
  Synthetic peptide
  MOD_RES (2)..(2): Nle
  MISC_FEATURE (3)..(3): X is (R)-alpha-methyl,alpha-octenylglycine
  MISC_FEATURE (10)..(10): X is (S)-alpha-methyl,alpha-pentenylglycine
  MISC_FEATURE (13)..(13): X is (R)-alpha-methyl,alpha-pentenylglycine
  MISC_FEATURE (17)..(17): X is (R)-alpha-methyl,alpha-pentenylglycine
  MISC_FEATURE (20)..(20): D-Glu
  MISC_FEATURE (21)..(21): D-Glu
AXXTYTGIFXDQXLSVXKGEE

SEQ ID NO: 23 - protein, 34 aa, Artificial Sequence
  Synthetic peptide
  MISC_FEATURE (15)..(15): X is (S)-alpha-methyl,alpha-pentenylglycine
  MISC_FEATURE (19)..(19): X is (S)-alpha-methyl,alpha-pentenylglycine
EKLRDLYSKSTAPAXSTYXGIFTDQVLSVLKGEE

SEQ ID NO: 24 - protein, 33 aa, Artificial Sequence
  Synthetic peptide
  MISC_FEATURE (25)..(25): X is (R)-alpha-methyl,alpha-octenylglycine
  MISC_FEATURE (32)..(23): X is (S)-alpha-methyl,alpha-pentenylglycine
  misc_feature (32)..(32): Xaa can be any naturally occurring amino acid
EKLRDLYSKSTAAMSTYTGIFTDQXLSVLKGXE

SEQ ID NO: 25 - protein, 33 aa, Artificial Sequence
  Synthetic peptide
  MISC_FEATURE (3)..(3): X is (R)-alpha-methyl,alpha-octenylglycine
  MISC_FEATURE (10)..(10): X is (S)-alpha-methyl,alpha-pentenylglycine
EKXRDLYSKXTAAFSTYFGIFTDQVLSVLKGEE

SEQ ID NO: 26 - protein, 33 aa, Artificial Sequence
  Synthetic peptide
  MISC_FEATURE (3)..(3): X is (R)-alpha-methyl,alpha-octenylglycine
  MISC_FEATURE (10)..(10): X is (S)-alpha-methyl,alpha-pentenylglycine
EKXRDLYSKXTAAFSTYTFIFTDQVLSVLKGEE

SEQ ID NO: 27 - protein, 21 aa, Artificial Sequence
  Synthetic peptide
  MOD_RES (2)..(2): Nle
  MISC_FEATURE (3)..(3): X is (R)-alpha-methyl,alpha-octenylglycine
  MISC_FEATURE (10)..(10): X is bis-pentenylglycine
  MISC_FEATURE (14)..(14): X is (R)-alpha-methyl,alpha-pentenylglycine
  MISC_FEATURE (20)..(20): D-Glu
  MISC_FEATURE (21)..(21): D-Glu
AXXTYTGIFXDQVXSVLKGEE

SEQ ID NO: 28 - protein, 21 aa, Artificial Sequence
  Synthetic peptide
  MOD_RES (2)..(2): Nle
  MISC_FEATURE (3)..(3): X is (S)-alpha-methyl,alpha-octenylglycine
  MISC_FEATURE (10)..(10): X is bis-pentenylglycine
  MISC_FEATURE (14)..(14): X is (S)-alpha-methyl,alpha-pentenylglycine
  MISC_FEATURE (20)..(20): D-Glu
  MISC_FEATURE (21)..(21): D-Glu
AXXTYTGIFXDQVXSVLKGEE

SEQ ID NO: 29 - protein, 21 aa, Artificial Sequence
  Synthetic peptide
  MOD_RES (2)..(2): Nle
  MISC_FEATURE (3)..(3): X is (R)-alpha-methyl,alpha-octenylglycine
  MISC_FEATURE (10)..(10): X is bis-pentenylglycine
  MISC_FEATURE (14)..(14): X is (R)-alpha-methyl,alpha-pentenylglycine
  MISC_FEATURE (18)..(18): X is alpha-methyl-Lysine
  MISC_FEATURE (20)..(20): D-Glu
  MISC_FEATURE (21)..(21): D-Glu
AXXTYTGIFXDQVXSVLXGEE

SEQ ID NO: 30 - protein, 21 aa, Artificial Sequence
  Synthetic peptide
  MOD_RES (2)..(2): Nle
  MISC_FEATURE (3)..(3): X is (S)-alpha-methyl,alpha-octenylglycine
  MISC_FEATURE (10)..(10): X is bis-pentenylglycine
  MISC_FEATURE (14)..(14): X is (S)-alpha-methyl,alpha-pentenylglycine
  MISC_FEATURE (18)..(18): X is alpha-methyl-Lysine
  MISC_FEATURE (20)..(20): D-Glu
  MISC_FEATURE (21)..(21): D-Glu
AXXTYTGIFXDQVXSVLXGEE

SEQ ID NO: 31 - protein, 20 aa, Artificial Sequence
  Synthetic peptide
  MISC_FEATURE (1)..(1): X is (R)-alpha-methyl,alpha-octenylglycine
  MISC_FEATURE (8)..(8): X is bis-pentenylglycine
  MISC_FEATURE (15)..(15): X is (S)-alpha-methyl,alpha-octenylglycine
  MOD_RES (17)..(17): MeGly
  MISC_FEATURE (18)..(18): D-Glu
  MISC_FEATURE (19)..(19): D-Glu
XTYTGIFXDQVLSVXKSSEE

SEQ ID NO: 32 - protein, 21 aa, Artificial Sequence
  Synthetic peptide
  MISC_FEATURE (1)..(1): D-Ala
  MOD_RES (2)..(2): D-Nle
  MISC_FEATURE (3)..(3): X is (R)-alpha-methyl,alpha-octenylglycine
  MISC_FEATURE (10)..(10): X is bis-pentenylglycine
  MISC_FEATURE (17)..(17): X is (S)-alpha-methyl,alpha-octenylglycine
  MOD_RES (19)..(19): MeGly
  MISC_FEATURE (20)..(20): D-Glu
  MISC_FEATURE (21)..(21): D-Glu
AXXTYTGIFXDQVLSVXKXEE

SEQ ID NO: 33 - protein, 22 aa, Artificial Sequence
  Synthetic peptide
  MISC_FEATURE (1)..(1): D-Ala
  MOD_RES (2)..(2): D-Nle
  MISC_FEATURE (3)..(3): X is (R)-alpha-methyl,alpha-octenylglycine
  MISC_FEATURE (10)..(10): X is bis-pentenylglycine
  misc_feature (11)..(11): Xaa can be any naturally occurring amino acid
  MISC_FEATURE (17)..(17): X is (S)-alpha-methyl,alpha-octenylglycine
  MISC_FEATURE (18)..(18): D-Lys
  MOD_RES (19)..(19): MeGly
  MISC_FEATURE (20)..(20): D-Glu
  MISC_FEATURE (21)..(21): D-Glu
AXXTYTGIFXXDQVLSVXKXEE

SEQ ID NO: 34 - protein, 21 aa, Artificial Sequence
  Synthetic peptide
  MISC_FEATURE (1)..(1): N-terminal acylation
  MOD_RES (2)..(2): Nle
  MISC_FEATURE (3)..(3): X is (R)-alpha-methyl,alpha-octenylglycine
  MISC_FEATURE (10)..(10): X is (S)-alpha-methyl,alpha-pentenylglycine
  MOD_RES (19)..(19): MeGly
  MISC_FEATURE (20)..(20): D-Glu
  MISC_FEATURE (21)..(21): D-Glu
AXXTYTGIFXDQVLSVLKXEE

SEQ ID NO: 35 - protein, 21 aa, Artificial Sequence
  Synthetic peptide
  MISC_FEATURE (1)..(1): D-Ala
  MOD_RES (2)..(2): D-Nle
  MISC_FEATURE (3)..(3): X is (R)-alpha-methyl,alpha-octenylglycine
  MISC_FEATURE (10)..(10): X is (S)-alpha-methyl,alpha-pentenylglycine
  MOD_RES (19)..(19): MeGly
  MISC_FEATURE (20)..(20): D-Glu
  MISC_FEATURE (21)..(21): D-Glu
AXXTYTGIFXDQVLSVLKXEE

SEQ ID NO: 36 - protein, 21 aa, Artificial Sequence
  Synthetic peptide
  MISC_FEATURE (1)..(1): D-Ala
  MOD_RES (2)..(2): D-Nle
  MISC_FEATURE (3)..(3): X is (R)-alpha-methyl,alpha-octenylglycine
  MISC_FEATURE (10)..(10): X is (S)-alpha-methyl,alpha-pentenylglycine
  MISC_FEATURE (18)..(18): D-Lys
  MOD_RES (19)..(19): MeGly
  MISC_FEATURE (20)..(20): D-Glu
  MISC_FEATURE (21)..(21): D-Glu
AXXTYTGIFXDQVLSVLKXEE

SEQ ID NO: 37 - protein, 22 aa, Artificial Sequence
  Synthetic peptide
  LIPID (1)..(1): N-terminal octanoic
  MOD_RES (2)..(2): Nle
  MISC_FEATURE (3)..(3): X is (R)-alpha-methyl,alpha-octenylglycine
  MISC_FEATURE (10)..(10): X is (S)-alpha-methyl,alpha-pentenylglycine
  misc_feature (11)..(11): Xaa can be any naturally occurring amino acid
  MOD_RES (19)..(19): MeGly
  MISC_FEATURE (20)..(20): D-Glu
  MISC_FEATURE (21)..(21): D-Glu
AXXTYTGIFXXDQVLSVLKXEE

SEQ ID NO: 38 - protein, 23 aa, Artificial Sequence
  Synthetic peptide
  LIPID (1)..(1): N-terminal octanoic acid
  MOD_RES (2)..(2): Nle
  MISC_FEATURE (3)..(3): X is (R)-alpha-methyl,alpha-octenylglycine
  MISC_FEATURE (10)..(10): X is (S)-alpha-methyl,alpha-pentenylglycine
  MOD_RES (19)..(19): MeGly
  MISC_FEATURE (20)..(20): D-GLu
  MISC_FEATURE (21)..(21): D-GLu
  MISC_FEATURE (22)..(22): D-GLu
  MISC_FEATURE (23)..(23): D-GLu
AXXTYTGIFXDQVLSVLKXEEEE

SEQ ID NO: 39 - protein, 21 aa, Artificial Sequence
  Synthetic peptide
  LIPID (1)..(1): N-terminal octanoic acid
  MISC_FEATURE (1)..(1): X is (R)-alpha-methyl,alpha-octenylglycine
  MISC_FEATURE (8)..(8): X is (S)-alpha-methyl,alpha-pentenylglycine
  MOD_RES (17)..(17): MeGly
  MISC_FEATURE (18)..(18): D-Glu
  MISC_FEATURE (19)..(19): D-Glu
  MISC_FEATURE (20)..(20): D-Glu
  MISC_FEATURE (21)..(21): D-Glu
XTYTGIFXDQVLSVLKXEEEE

SEQ ID NO: 40 - protein, 23 aa, Artificial Sequence
  Synthetic peptide
  MISC_FEATURE (1)..(1): N-terminal Acylation
  MOD_RES (2)..(2): Nle
  MISC_FEATURE (3)..(3): X is (R)-alpha-methyl,alpha-octenylglycine
  MISC_FEATURE (10)..(10): X is (S)-alpha-methyl,alpha-pentenylglycine
  MOD_RES (19)..(19): MeGly
  MISC_FEATURE (20)..(20): D-Glu
  MISC_FEATURE (21)..(21): D-Glu
  MISC_FEATURE (22)..(22): D-Glu
  MISC_FEATURE (23)..(23): D-Glu
AXXTYTGIFXDQVLSVLKXEEEE

SEQ ID NO: 41 - protein, 21 aa, Artificial Sequence
  Synthetic peptide
  MOD_RES (2)..(2): Nle
  MISC_FEATURE (3)..(3): X is (R)-alpha-methyl,alpha-octenylglycine
  MISC_FEATURE (10)..(10): X is bis-pentenylglycine
  MISC_FEATURE (17)..(17): X is (S)-alpha-methyl,alpha-octenylglycine
  MOD_RES (19)..(19): MeGly
  MISC_FEATURE (20)..(20): D-Glu
  MISC_FEATURE (21)..(21): D-Glu
AXXTYTGIFXDQVLSVXKXEE

SEQ ID NO: 42 - protein, 19 aa, Artificial Sequence
  Synthetic peptide
  MISC_FEATURE (1)..(1): N-terminal octanoic acid
  MISC_FEATURE (1)..(1): X is (R)-alpha-methyl,alpha-octenylglycine
  MISC_FEATURE (8)..(8): X is bis-pentenylglycine
  MISC_FEATURE (15)..(15): X is bis-pentenylglycine (S)-alpha-methyl, alpha-octenylglycine
  MOD_RES (17)..(17): MeGly
  MISC_FEATURE (18)..(18): D-Glu
  MISC_FEATURE (19)..(19): D-Glu
XTYTGIFXDQVLSVXKXEE

SEQ ID NO: 43 - protein, 21 aa, Artificial Sequence
  Synthetic peptide
  MISC_FEATURE (1)..(1): N-terminal octanoic acid
  MOD_RES (1)..(1): Aib
  MOD_RES (2)..(2): Nle
  MISC_FEATURE (3)..(3): X is (R)-alpha-methyl,alpha-octenylglycine
  MISC_FEATURE (10)..(10): X is bis-pentenylglycine
  MISC_FEATURE (17)..(17): X is (S)-alpha-methyl,alpha-octenylglycine
  MOD_RES (19)..(19): MeGly
  MISC_FEATURE (20)..(20): D-Glu
  MISC_FEATURE (21)..(21): D-Glu
XXXTYTGIFXDQVLSVXKXEE

SEQ ID NO: 44 - protein, 79 aa, Artificial Sequence
  Syntthetic peptide
TQQPQQDEMPSPTFLTQVKESLSSYWESAKTAAQNLYEKTYLPAVDEKLRDLYSKSTAAMSTYTGIFTDQVLSVLKGEE

SEQ ID NO: 45 - protein, 13 aa, Artificial Sequence
  Synthetic peptide
  MISC_FEATURE (3)..(3): X is penicillamine
  MOD_RES (9)..(9): MeGly
  MOD_RES (10)..(10): N-methylglycine
  MISC_FEATURE (12)..(12): C-terminal 6-aminohexanoic acid
RFXTGHFGXXYPC

SEQ ID NO: 46 - protein, 15 aa, Artificial Sequence
  Synthetic peptide
  MISC_FEATURE (15)..(15): C-terminal 6-aminohexanoic acid
VMHCFWDEEFKCDYG

(19) United States
(12) Patent Application Publication
(10) Pub. No.: US 2024/0309065 A1
(43) Pub. Date: Sep. 19, 2024

(54) SHORT APOC-II MIMETIC PEPTIDES AND METHODS OF USE

(71) Applicant: The United States of America, as represented by the Secretary, Dept. of Health and Human Services, Bethesda, MD (US)

(72) Inventors: Alan T. Remaley, Bethesda, MD (US); Denis Sviridov, Rockville, MD (US); Mart Reimund, Rockville, MD (US); Anna Wolska, Kensington, MD (US); Amaury Dasseux, Bethesda, MD (US)

(73) Assignee: The United States of America, as represented by the Secretary, Dept. of Health and Human Services, Bethesda, MD (US)

(21) Appl. No.: 18/572,462

(22) PCT Filed: Jun. 22, 2022
(86) PCT No.: PCT/US2022/034511

§ 371 Date: Dec. 20, 2023

Related U.S. Application Data
(60) Provisional application No. 63/214,081, filed on Jun. 23, 2021.

(51) Int. Cl. C07K 14/775; A61K 38/00; A61P 3/06; C07K 14/00

(52) U.S. Cl. CPC C07K 14/775; A61P 3/06; C07K 14/001; A61K 38/00

(57) ABSTRACT

ApoC-II mimetic peptides of 19-35 amino acids including one or two helical domains with one or more covalent linkages joining at least two non-contiguous amino acids of the peptide, wherein at least one of the helical domains is an amphipathic helical domain, are provided. Methods of treating dyslipidemic disorders, such as hypertriglyceridemia or hypercholesterolemia, using the peptides are also provided.

### CROSS REFERENCE TO RELATED APPLICATIONS

This application claims the benefit of U.S. Provisional Patent Application No. 63/214,081 filed Jun. 23, 2021, which is incorporated herein by reference in its entirety.

### FIELD

This disclosure relates to apolipoprotein C-II (apoC-II) mimetic peptides and methods of their use, particularly for treating hypertriglyceridemia and/or hypercholesterolemia.

### BACKGROUND

Hypertriglyceridemia is typically defined as serum triglyceride levels over 150 mg/dL. Hypertriglyceridemia is most commonly secondary to obesity, diabetes mellitus, pregnancy, alcohol and a wide variety of drugs, but is also sometimes the result of genetic defects in lipoprotein lipase (LPL) or apoC-II, an obligate activator of LPL. Patients with moderate increases in triglycerides are at risk for cardiovascular diseases and severe hypertriglyceridemia also increases risk for acute pancreatitis.

Triglycerides are enriched in the core of chylomicrons and very low-density lipoproteins (VLDL). Lipolysis by LPL is a critical step in the catabolism of these particles. Fibrates and supplements rich in omega-3 polyunsaturated fatty acids, such as fish oils, are the main treatments for hypertriglyceridemia; however, it is not clear that these treatments lower the risks associated with elevated triglycerides, such as cardiovascular diseases. Therefore, new therapeutic agents for treatment of hypertriglyceridemia are needed.

### SUMMARY

Disclosed herein are ApoC-II mimetic peptides. In some embodiments, the peptides include an isolated ApoC-II mimetic peptide of 19-35 amino acids including one or two helical domains with one or more covalent linkages joining at least two non-contiguous amino acids of the peptide, wherein at least one of the helical domains is an amphipathic helical domain. In embodiments, the covalent linkage joining at least two non-contiguous amino acids is between two amino acids on the hydrophobic side of the amphipathic helical domain. In some examples, the one or more covalent linkages include one of more of a hydrocarbon staple, a hydrocarbon stitch, a lactam bridge, a disulfide bond, or a combination of two or more thereof. In some embodiments, the linkage is a hydrocarbon staple or hydrocarbon stitch, which includes a linkage including one or more of (S)-α-methyl, α-pentenylglycine (S5), (S)-α-methyl, α-octenylglycine (S8), bis-pentenylglycine (B5), (R)-α-methyl, α-pentenylglycine (R5), and (R)-α-methyl, α-octenylglycine (R8).

In some embodiments, the disclosed peptides include one or more additional modifications. In some examples, the modification includes one or more of amino acid substitutions, additions, or deletions; C-terminal amidation; N-terminal acylation; one or more D-isomer amino acids; a modified or non-natural amino acid; an N-terminal fatty acid; a C-terminal fatty acid; or a combination of two or more thereof. In particular examples, the peptide includes an octanoic acid or myristic acid modification at the N- or C-terminus. In other examples, the modification is a human neonatal Fc receptor (FcRn) binding sequence, such as an IgG binding site (e.g., SEQ ID NO: 45) or an albumin binding site (e.g., SEQ ID NO: 46).

In some embodiments, the disclosed peptides include an amino acid sequence with at least 90% identity to the amino acid sequence of any one of SEQ ID NOs: 1-11 and 16-43. In other examples, the peptides include or consist of the amino acid sequence of any one of SEQ ID NOs: 1-11 and 16-43. In particular examples, the peptide is 19-25 amino acids long.

In some embodiments, the peptide activates lipoprotein lipase and/or displaces ApoC-III from very low density lipoprotein. In additional embodiments, the disclosed peptides have one or more improved properties (for example, compared to a peptide lacking the one or more covalent linkages), including stabilization of alpha helical structure, improved LPL activation, improved ability to displace apoC-III from lipoproteins, improved binding to lipoproteins, increased resistance to proteolysis, or a combination of two or more thereof.

Also provided are pharmaceutical compositions including a disclosed ApoC-II mimetic peptide and a pharmaceutically acceptable carrier. In some embodiments, the composition is formulated for intravenous administration, subcutaneous administration, or oral administration.

In some embodiments, methods of decreasing triglyceride levels in a subject are provided, the methods including administering to the subject an effective amount of a disclosed peptide or composition. In some examples, the subject has hypertriglyceridemia or lipoprotein lipase deficiency. In one example, the subject has a pre-treatment serum triglyceride level of 150 mg/dL or more.

In other embodiments, methods of decreasing cholesterol levels in a subject are provided, the methods including administering to the subject an effective amount of a disclosed peptide or composition. In some examples, the subject has hypercholesteremia.

The methods of decreasing triglyceride levels and/or cholesterol levels in a subject in some examples include intravenous administration, subcutaneous injection, or oral administration of a disclosed peptide or composition. In some embodiments, the subject is human.

Also provided are methods of making the disclosed peptides, which in some embodiments include producing the peptide recombinantly, for example by chemical synthesis.

The foregoing and other features of the disclosure will become more apparent from the following detailed description, which proceeds with reference to the accompanying figures.

### BRIEF DESCRIPTION OF THE DRAWINGS

FIGS. 1A-1C are a series of schematic diagrams showing the ApoC-II region selected for stapled peptides. FIG. 1A shows the amino acid sequence of human ApoC-II protein (SEQ ID NO: 44) and the region selected for preparing stapled peptides, including residues important for LPL activation. FIG. 1B is a helical wheel plot of the third helix of ApoC-II starting with residue A59 (SEQ ID NO: 14). FIG. 1C illustrates exemplary reagents used for short and long hydrocarbon staples.

FIG. 2 is a graph showing the effect of a long hydrocarbon staple and addition of a proline between two helices on LPL activation. 1. Control (ApoC-II; SEQ ID NO: 13); 16. CP (SEQ ID NO: 12); D6-PV (SEQ ID NO: 15); 2. CS-new (SEQ ID NO: 1); 12. HIS-new (SEQ ID NO: 6).

FIG. 3 is a graph showing the effect of a short hydrocarbon staple and addition of a proline between two helices on LPL activation. 1. Control (ApoC-II; SEQ ID NO: 13); 16. CP (SEQ ID NO: 12); D6-PV (SEQ ID NO: 15); 3. H2S (SEQ ID NO: 2); 15. H2SP (SEQ ID NO: 23).

FIG. 4 is a graph showing the effect of a long hydrocarbon staple vs. a short hydrocarbon staple on LPL activation. 1. Control (ApoC-II; SEQ ID NO: 13); 2. CS-new (SEQ ID NO: 1); 3. H2S (SEQ ID NO: 2); 6. H2LSL (SEQ ID NO: 3); 7. HSLSR (SEQ ID NO: 4).

FIG. 5 is a graph showing the effect of location of the hydrocarbon staple on LPL activation. 1. Control (ApoC-II; SEQ ID NO: 13); 2. CS-new (SEQ ID NO: 1); D6-PV (SEQ ID NO: 15); 8. H2TS (SEQ ID NO: 24).

FIG. 6 is a graph showing the effect of substitution to a phenylalanine residue in the second helix on LPL activation. 1. Control (ApoC-II; SEQ ID NO: 13); 2. CS-new (SEQ ID NO: 1); D6-PV (SEQ ID NO: 15); 4. HISH2F6064 (SEQ ID NO: 25); 5. HISH2F6065 (SEQ ID NO: 26).

FIG. 7 is a graph showing the effect of peptide length on LPL activation. 19. ApoC-II truncated (SEQ ID NO: 14); D6-PV (SEQ ID NO: 15); 7. H2LSR (SEQ ID NO: 4); 9. SPH2S (SEQ ID NO: 5).

FIGS. 8A and 8B show LPL activation assay over extended concentrations for selected peptides. FIG. 8A shows linear scale and FIG. 8B shows logarithmic scale. CS-new (SEQ ID NO: 1); HSLSR (SEQ ID NO: 3); SPH2S (SEQ ID NO: 5); D6-PV (SEQ ID NO: 15).

FIG. 9 shows displacement of ApoC-III from VLDL by peptides. D6-PV (SEQ ID NO: 15); SPH2S (SEQ ID NO: 5); H2LSR (SEQ ID NO: 4); CS-new (SEQ ID NO: 1).

FIGS. 10A and 10B show LPL activation assay over extended concentrations for additional peptides. FIG. 10A shows linear scale and FIG. 10B shows logarithmic scale. 1. SSP-CTB (SEQ ID NO: 9); 2. SSP-T1 (SEQ ID NO: 7); 3. SSP-T2 (SEQ ID NO: 8); 4. SSP-SS (SEQ ID NO: 11); 5. SSP-dE (SEQ ID NO: 10); 6. SPH2S (SEQ ID NO: 5).

FIGS. 11A and 11B show absolute plasma triglycerides (FIG. 11A) and percent baseline (FIG. 11B) in ApoC-II-KO mice treated as indicated. Tris (control); D6-PV (SEQ ID NO: 15); SPH2S (SEQ ID NO: 5).

FIGS. 12A and 12B show absolute total plasma cholesterol (TC, FIG. 12A) and percent baseline total cholesterol (FIG. 12B) in ApoC-II-KO mice treated as indicated. Tris (control); D6-PV (SEQ ID NO: 15); SPH2S (SEQ ID NO: 5).

FIGS. 13A and 13B show absolute total plasma non-esterified fatty acids (NEFA; FIG. 13A) and percent baseline NEFA (FIG. 13B) in ApoC-II-KO mice treated as indicated. Tris (control); D6-PV (SEQ ID NO: 15); SPH2S (SEQ ID NO: 5).

FIGS. 14A-14C show in vitro LPL assays with the indicated peptides. FIG. 14A: SSP-T1 (SEQ ID NO: 7); SPHS2-Sarc (SEQ ID NO: 34); SPH2S-Sarc-Da (SEQ ID NO: 35); and SPH2S-Sarc-Dak (SEQ ID NO: 36). FIG. 14B: SPLLee-Sarc (SEQ ID NO: 18); SPLLee-Sarc-19 (SEQ ID NO: 31); SPLLee-Sarc-Da (SEQ ID NO: 32); and SPLLee-Sarc-Dak (SEQ ID NO: 33). FIG. 14C: Peptide 1 (Octa-SPH2S-Sarc; SEQ ID NO: 37); Peptide 2 (Octa-SPH2S-Sarc-4e; SEQ ID NO: 38); Peptide 3 (Octa-SPH2S-Sarc-19-4e; SEQ ID NO: 39); Peptide 4 (Ac-SPH2S-Sarc-19-4e; SEQ ID NO: 40); Peptide 5 (SPH2S-Sarc; SEQ ID NO: 34); and peptide 6 (SPLLee-Sarc; SEQ ID NO: 18).

FIG. 15 is a graph showing DMPC vesicle solubilization assay. The results are presented as percentage of baseline. ApoC2-helix 3 (SEQ ID NO: 14); D6-PV (SEQ ID NO: 15); SPH2S-Sarc (SEQ ID NO: 34; SPL33-Sarc (SEQ ID NO: 18).

FIGS. 16A-16D are graphs showing proteolysis of the indicated peptides by trypsin and proteinase K (as percent of baseline). FIG. 16A: SSP-T1 (SEQ ID NO: 7). FIG. 16B: SPH2S-Sarc (SEQ ID NO: 34). FIG. 16C: SPH2S-Sarc-Da (SEQ ID NO: 35). FIG. 16D: SPLLee-Sarc (SEQ ID NO: 18).

FIG. 17 is a graph showing LPL activation of the indicated peptides after pepsin treatment (as percent of baseline). D6 PV (SEQ ID NO: 15); SPH2S-Sarc (SEQ ID NO: 34); SPLLee-Sarc (SEQ ID NO: 18).

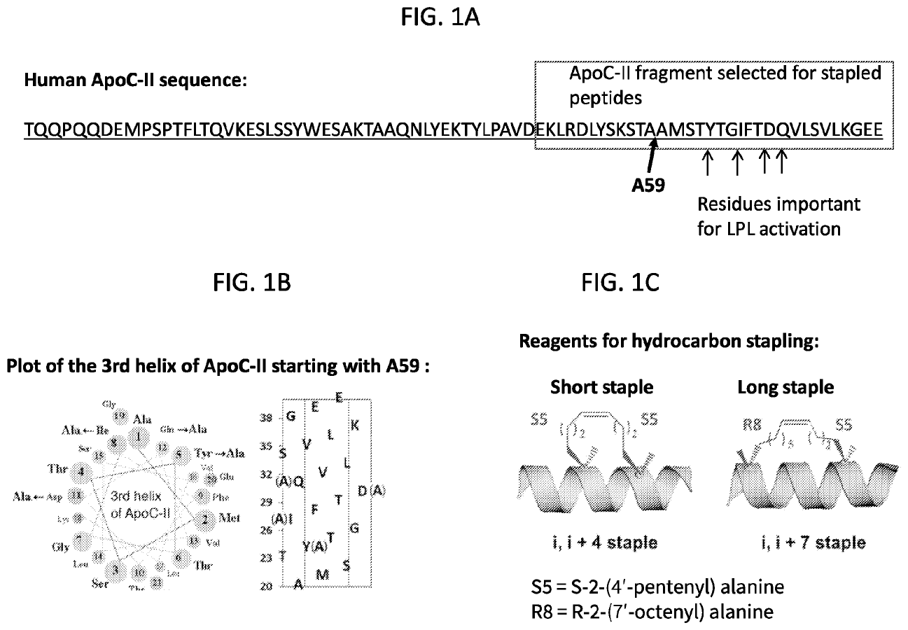

### SEQUENCE LISTING

Any nucleic acid and amino acid sequences provided herein and in the accompanying Sequence Listing are shown using standard letter abbreviations for nucleotide bases and amino acids, as defined in 37 C.F.R. § 1.822. In at least some cases, only one strand of each nucleic acid sequence is shown, but the complementary strand is understood as included by any reference to the displayed strand.

SEQ ID NOs: 1-11 are amino acid sequences of exemplary ApoC-II fragment stapled peptides.

SEQ ID NO: 12 is the amino acid sequence of a proline modified ApoC-II peptide.

SEQ ID NO: 13 is the amino acid sequence of an ApoC-II peptide.

SEQ ID NO: 14 is the amino acid sequence a truncated ApoC-II peptide.

SEQ ID NO: 15 is the amino acid sequence of D6PV peptide.

SEQ ID NOs: 16-43 are the amino acid sequences of additional exemplary ApoC-II fragment stapled or stitched peptides.

SEQ ID NO: 44 is the amino acid sequence of an exemplary mature human ApoC-II protein.

SEQ ID NO: 45 is an exemplary human neonatal Fc receptor (FcRn) IgG binding site: RF(penicillamine)TGHFG(N-methyl-Glycine)(N-methyl-Leucine)YPC-6-aminohexanoic acid

SEQ ID NO: 46 is an exemplary human neonatal Fc receptor (FcRn) albumin binding site:

VMHCFWDEEFKCDYG-6-aminohexanoic acid

### DETAILED DESCRIPTION

### I. Terms

Unless otherwise explained, all technical and scientific terms used herein have the same meaning as commonly understood by one of ordinary skill in the art to which this disclosure belongs. Definitions of common terms in molecular biology may be found in Benjamin Lewin, Genes X, published by Jones & Bartlett Publishers, 2009; and Meyers et al. (eds.), The Encyclopedia of Cell Biology and Molecular Medicine, published by Wiley-VCH in 16 volumes, 2008; and other similar references.

The singular terms “a,” “an,” and “the” include plural referents unless the context clearly indicates otherwise. Similarly, the word “or” is intended to include “and” unless the context clearly indicates otherwise. Hence “comprising A or B” means including A, or B, or A and B. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

In order to facilitate review of the various embodiments of the disclosure, the following explanations of specific terms are provided:

Alkyl: Refers to a saturated aliphatic hydrocarbyl group having from 1 to 25 (C1-25) or more carbon atoms, such as from 1 to 10 (C₁₋₁₀) carbon atoms, from 1 to 4 (C₁₋₄) carbon atoms, from 1 to 14 (C₁₋₁₄) carbon atoms, or from 2 to 22 (C₂₋₂₂) carbon atoms or from 6 to 18 (C₆₋₁₈) carbon atoms. An alkyl moiety may be substituted or unsubstituted. This term includes, by way of example, linear and branched hydrocarbyl groups such as methyl (CH₃), ethyl (—CH₂CH₃), n-propyl (—CH₂CH₂CH₃), isopropyl (—CH(CH₃)₂), n-butyl (—CH₂CH₂CH₂CH₃), isobutyl (—CH₂CH₂(CH₃)₂), sec-butyl (—CH(CH₃)(CH₂CH₃), t-butyl (—C(CH₃)₃), n-pentyl (—CH₂CH₂CH₂CH₂CH₃), neopentyl (—CH₂C(CH₃)₃), hexyl (C₆H₁₃), heptyl (CH₁₅), octyl (C₈H₁₇), decyl (C₁₀H₂₁), dodecyl (C₁₂H₂₅), tetradecyl (C₁₄H₂₉), hexadecyl (C₁₆H₃₃), octadecyl (C₁₈H₃₇) or eicosanyl (C₂₀H₄₁).

Amphipathic: An amphipathic molecule contains both hydrophobic (non-polar) and hydrophilic (polar) groups. The hydrophobic group can be an alkyl group, such as a long carbon chain, for example, with the formula: CH₃(CH₂)ₙ, (where n is generally greater than or equal to about 4 to about 16). Such carbon chains also optionally comprise one or more branches, wherein a hydrogen is replaced with an aliphatic moiety, such as an alkyl group. A hydrophobic group also can comprise an aryl group. The hydrophilic group can be one or more of the following: an ionic molecule, such as an anionic molecule (e.g., a fatty acid, a sulfate or a sulfonate) or a cationic molecule, an amphoteric molecule (e.g., a phospholipid), or a non-ionic molecule (e.g., a small polymer).

One example of an amphipathic molecule is an amphipathic peptide. An amphipathic peptide can also be described as a helical peptide that has hydrophilic amino acid residues on one face of the helix and hydrophobic amino acid residues on the opposite face. In some examples, peptides described herein will form amphipathic helices in vitro or in vivo under appropriate conditions.

Apolipoprotein C-II (apoC-II): A 79 amino acid protein, which plays a role in plasma lipid metabolism as an activator of lipoprotein lipase (LPL). This protein includes three amphipathic helices: helix 1, residues 16-38; helix 2, residues 45-58; and helix 3, residues 64-74. The lipase-activating region of apoC-II has previously been localized to the C-terminal domain of the sequence, from about residue 56, whereas the N-terminal domain (residues 1-50) of the sequence is involved in lipid binding, thus allowing the protein to anchor to the surface of lipoprotein particle.

Unless the context clearly indicates otherwise, the term ApoC-II includes any apoC-II gene, cDNA, mRNA, or protein from any organism and is capable of activating lipoprotein lipase. Nucleic acid and protein sequences for ApoC-II are publicly available. For example, GenBank Accession No. NM_009695 (human) discloses an apoC-II nucleic acid sequence, and GenBank Accession Nos. AAH05348 (human), NP_001078821 (rat), NP_001095850 (bovine), and NP_033825 (mouse) disclose ApoC-II protein sequences, all of which are incorporated by reference as provided by GenBank on May 20, 2021. In other examples, ApoC-II includes the mature (processed) form of the protein, for example, lacking the signal sequence. SEQ ID NO: 44 is the amino acid sequence of an exemplary mature human ApoC-II protein.

Domain: A domain of a protein is a portion or fragment of a protein that shares common structural, physiochemical and functional features; for example, hydrophobic, polar, globular, helical domains or properties, for example an alpha helical domain, such as an amphipathic helical domain.

Dyslipidemic disorder: A disorder associated with an altered amount of any or all of the lipids or lipoproteins in the blood. Dyslipidemic disorders include, for example, hyperlipidemia, hyperlipoproteinemia, hypercholesterolemia, hypertriglyceridemia, HDL deficiency, apoA-I deficiency, and cardiovascular disease (e.g., coronary artery disease, atherosclerosis and restenosis).

Hydrocarbon Staple or Stitch: A carbon-carbon bond between at least two moieties in a peptide, which in some examples, stabilizes an alpha-helical structure. Hydrocarbon staples are all-hydrocarbon cross-links formed from α,α-disubstituted non-natural amino acids that include terminal olefin tethers. In some examples, the non-natural amino acids are non-contiguous, for example, separated by 3 or 6 amino acids. Hydrocarbon stitching refers to formation of two hydrocarbon staples with a common attachment point at the middle position (e.g., formation of two staples using three modified amino acids). Exemplary non-natural amino acids that can be used to form hydrocarbon staples or stitches include (S)-α-methyl, α-pentenylglycine (S5), (S)-α-methyl, α-octenylglycine (S8), (R)-α-methyl, α-pentenylglycine (R5), and (R)-α-methyl, α-octenylglycine (R8), in any combination. In addition, bis-pentenylglycine (B5) can be used to form hydrocarbon stitches, for example, allowing formation of two staples with B5 serving as the common attachment point. Hydrocarbon staples and stitches (which contain adjacent staples) are described in e.g., Walensky and Bird, J. Med. Chem. 57:6275-6288, 2014 and Hilinski et al., J. Am. Chem. Soc. 136:12314-12322, 2014, both of which are incorporated herein by reference in their entirety.

Inhibiting or treating a disease: Inhibiting the full development of a disease, disorder or condition, for example, in a subject who is at risk for a disease such as dyslipidemic disorder. “Treatment” refers to a therapeutic intervention that ameliorates a sign or symptom of a disease or pathological condition after it has begun to develop. As used herein, the term “ameliorating,” with reference to a disease, pathological condition or symptom, refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, a reduction in the number of relapses of the disease, an improvement in the overall health or well-being of the subject, or by other parameters known in the art that are specific to the particular disease.

Isolated: An “isolated” biological component (such as a nucleic acid molecule, protein or organelle) is one that has been substantially separated or purified away from other biological components in the cell of the organism in which the component naturally occurs, e.g., other chromosomal and extra-chromosomal DNA and RNA, proteins and organelles. Nucleic acids and proteins that have been “isolated” include nucleic acids and proteins purified by standard purification methods. The term also embraces nucleic acids and proteins prepared by recombinant expression in a host cell as well as chemically synthesized nucleic acids.

Linker or Linkage: A molecule that joins two other molecules, either covalently, or through ionic, van der Waals or hydrogen bonds. In particular examples, a linker comprises a hydrocarbon staple or hydrocarbon stitch. In other examples, a linker comprises a lactam bridge or a disulfide bond.

Lipoprotein: A biochemical assembly that contains both proteins and lipids, bound to the proteins, which allows fats to move through the water inside and outside cells. There are five major groups of lipoprotein particles, which, in order of molecular size, largest to smallest, are chylomicrons, very low-density lipoprotein (VLDL), intermediate-density lipoprotein (IDL), low-density lipoprotein (LDL), and high-density lipoprotein (HDL). HDL contains the highest proportion of protein to cholesterol; its most abundant apolipoproteins are ApoA-I and ApoA-II. LDL contains apolipoprotein B, and has a core consisting of linoleate and includes esterified and non-esterified cholesterol molecules. LDL particles are approximately 22 nm in diameter and have a mass of about 3 million Daltons. Lipoprotein a, (Lp(a)) is a lipoprotein subclass; lipoprotein a consists of an LDL-like particle and the specific apolipoprotein(a) (apo(a)), which is covalently bound to the apolipoprotein B of the LDL-like particle.

Lipoprotein lipase (LPL): An enzyme that degrades circulating triglycerides in the blood stream (such as triglycerides embedded in VLDL). It is present on the vascular endothelial surface, anchored to capillary walls. LPL also functions as a ligand/bridging factor for receptor-mediated lipoprotein uptake. ApoC-II is a cofactor for LPL. Mutations that cause LPL deficiency result in type I hyperlipoproteinemia and mutations that cause less severe effects on LPL are linked to disorders of lipoprotein metabolism.

Peptide: A polymer in which the monomers are amino acid residues which are joined together, e.g., through amide bonds. When the amino acids are alpha-amino acids, either the L-optical isomer or the D-optical isomer can be used. The terms “peptide” or “polypeptide” as used herein are intended to encompass any amino acid sequence and include modified sequences such as glycoproteins. The term “peptide” is specifically intended to cover naturally occurring peptides, as well as those which are recombinantly or synthetically produced. “Amino acid” refers to naturally occurring alpha amino acids, unnatural alpha amino acids, natural beta amino acids, and unnatural beta amino acids, as well as modified amino acids (for example, modified by addition of a carbohydrate group, a hydroxyl group, a phosphate group, a fatty acid group, a linker, or a functional group). The term “residue” or “amino acid residue” includes reference to an amino acid that is incorporated into a peptide, polypeptide, or protein.

Unnatural amino acids include without limitation 4-hydroxyproline, desmosine, gamma-aminobutyric acid, beta-cyanoalanine, norvaline, 4-(E)-butenyl-4(R)-methyl-N-methyl-L-threonine, N-methyl-L-leucine, 1-amino-cyclopropanecarboxylic acid, 1-amino-2-phenyl-cyclopropanecarboxylic acid, 1-amino-cyclobutanecarboxylic acid, 4-amino-cyclopentenecarboxylic acid, 3-amino-cyclohexanecarboxylic acid, 4-piperidylacetic acid, 4-amino-1-methylpyrrole-2-carboxylic acid, 2,4-diaminobutyric acid, 2,3-diaminopropionic acid, 2,4-diaminobutyric acid, 2-aminoheptanedioic acid, 4-(aminomethyl)benzoic acid, 4-aminobenzoic acid, ortho-, meta- and para-substituted phenylalanines, disubstituted phenylalanines, substituted tyrosines, and statine. Additionally, amino acids may be derivatized to include amino acid residues that are hydroxylated, phosphorylated, sulfonated, acylated, or glycosylated.

Pharmaceutically acceptable carriers: Pharmaceutically acceptable carriers are known to one of ordinary skill in the art. For example, Remington: The Science and Practice of Pharmacy, 22^{nd} ed., London, UK: Pharmaceutical Press (2013), describes compositions and formulations suitable for pharmaceutical delivery of the peptides disclosed herein.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Purified: The term purified does not require absolute purity; rather, it is intended as a relative term. Thus, for example, a purified compound preparation is one in which the compound is more enriched than the compound is in its generative environment, for instance within a cell or in a biochemical reaction chamber. In some embodiments, a preparation of compound is purified such that the compound represents at least 50% of the content of the preparation.

Recombinant: A recombinant nucleic acid or protein is one that has a sequence that is not naturally occurring or has a sequence that is made by an artificial combination of two otherwise separated segments of sequence. This artificial combination can be accomplished by chemical synthesis or, more commonly, by the artificial manipulation of isolated segments of nucleic acids, e.g., by genetic engineering techniques. In some examples, a recombinant protein is one encoded for by a recombinant nucleic acid molecule.

Subject: A living multi-cellular vertebrate organism, a category that includes both human and veterinary subjects, including human and non-human mammals.

Therapeutically effective amount: A quantity of a specified agent (or combination of agents) sufficient to achieve a desired effect in a subject being treated with that agent.

### II. ApoC-II Mimetic Peptides

Disclosed herein are ApoC-II mimetic peptides. The peptides are 19-35 amino acids long and include one or two helical domains with one or more covalent linkages joining at least two non-contiguous amino acids of the peptide, wherein at least one of the helical domains is an amphipathic helical domain. In some embodiments, the ApoC-II mimetic peptides increase LPL activity, decrease ApoC-III binding to VLDL, reduce serum triglyceride levels in a subject, reduce total serum cholesterol levels in a subject, or a combination of two or more thereof.

The disclosed ApoC-II mimetic peptides include the third helical domain of ApoC-II (such as amino acids 59-79 of SEQ ID NO: 44). In some embodiments, the disclosed ApoC-II mimetic peptides include the second and third helical domains of ApoC-II (such as amino acids 47-79 of SEQ ID NO: 44). In some embodiments the peptide includes an ApoC-II peptide having one or more modifications including addition, deletion, and/or substitution of one or more amino acids compared with a naturally occurring ApoC-II amino acid sequence (such as SEQ ID NOs: 13 or 14).

The peptides include one or more (such as 1, 2, 3, or 4) covalent linkages between two non-contiguous amino acids in the peptide. In some embodiments, the peptide includes one covalent linkage between a pair of non-contiguous amino acids in the peptide. In other embodiments, the peptide includes two covalent linkages, each covalent linkage being between a different pair of non-contiguous amino acids in the peptide. In still further embodiments, the peptide includes two covalent linkages between three non-contiguous amino acids in the peptide, wherein one of the non-contiguous amino acids has a covalent linkage to each of the other two non-contiguous amino acids. In some embodiments, the covalent linkage is a hydrocarbon staple, a hydrocarbon stitch, a disulfide bond, or a lactam bridge (e.g., formed by cyclization of lysine ε-amino groups with glutamic or aspartic acid side group carboxyl groups) or other chemical linkages that stabilize helix formation between the hydrocarbon staples.

When two or more covalent linkages are present, the covalent linkages may be the same or different covalent linkages. In some specific examples, a disclosed peptide includes one hydrocarbon staple, two hydrocarbon staples, one hydrocarbon stitch (comprising two hydrocarbon staples), or one hydrocarbon staple and one lactam bridge. In some examples, the covalent linkage(s) provide one or more improved properties to the peptide, including stabilization of alpha helical structure, improved LPL activation, improved ability to displace apoC-III from lipoproteins, improved binding to lipoproteins, and resistance to proteolysis.

In particular embodiments, the disclosed peptides include one or more hydrocarbon staples or hydrocarbon stitches. A hydrocarbon staple is an all-hydrocarbon cross-link formed between two moieties, such as α,α-disubstituted non-natural amino acids that include terminal olefin tethers. A hydrocarbon stitch is made up of two hydrocarbon staples with a common attachment point at a middle position. Thus, in some examples, at least two non-contiguous amino acids in the ApoC-II peptide are replaced with non-natural amino acids that are capable of forming a hydrocarbon staple when linked. In some examples, the non-contiguous amino acids are separated by 3 or 6 amino acids.

Exemplary non-natural amino acids that can be used to form hydrocarbon staples or stitches include (S)-α-methyl, α-pentenylglycine (S5), (S)-α-methyl, α-octenylglycine (S8), (R)-α-methyl, α-pentenylglycine (R5), (R)-α-methyl, α-octenylglycine (R8), bis-pentenylglycine (B5), (S)-pentenylalanine, (S)-octenylalanine, (R)-pentenylalanine, and (R)-octenylalanine, in any combination. In some examples, the non-natural amino acids are selected such that they are capable of forming a hydrocarbon staple. Thus, for example, if the two non-natural amino acids are separated by 2 or 3 amino acids, the hydrocarbon staple is “short” and the linkage is between two pentenyl-modified amino acids (such as between S5 and S5, between R5 and R5, between S5 and R5, or between R5 and S5). If the two non-natural amino acids are separated by 6 amino acids, the hydrocarbon staple is “long” and the linkage is between a pentyl-modified amino acid and an octenyl-modified amino acid (such as between R8 and S5 or between S8 and R5). In other examples, the non-natural amino acids are selected such that they are capable of forming a hydrocarbon stitch. In one specific example a hydrocarbon stitch is formed by including an octenyl-modified amino acid separated from a bis-pentenylglycine residue by 6 amino acids, which in turn is separated from a pentenyl-modified amino acid by 3 amino acids (such as R8-B5-R5, R8-B5-S5, S8-B5-S5, or S8-B5-R5). In other examples, a hydrocarbon stitch is formed by including a pentynyl-modified amino acid separated from a bis-pentenylglycine residue by 3 amino acids, which in turn is separated from an octenyl-modified amino acid by 6 amino acids (such as S5-B5-S8, S5-B5-R8, R5-B5-R8, or R5-B5-S8). In another example, a hydrocarbon stitch is formed by including an octenyl-modified amino acid separated from a bis-pentenylglycine residue by 6 amino acids, which in turn is separated from a second octenyl-modified amino acid by 6 amino acids (such as R8-B5-S8, S8-B5-R8, R8-B5-R8, or S8-B5-S8). In yet another example, a hydrocarbon stitch is formed by including a pentynyl-modified amino acid separated from a bis-pentenylglycine residue by 3 amino acids, which in turn is separated from an pentynyl-modified amino acid by 3 amino acids (such as R5-B5-S5, R5-B5-R5, S5-B5-R5, or S5-B5-S5).

Also encompassed by the present disclosure are peptides including one or more additional modifications in addition to the covalent linkages discussed above and any modifications required to create such linkages. In some embodiments the peptide includes one or more modifications including addition, deletion, and/or substitution of one or more amino acids in the ApoC-II mimetic peptides with another naturally occurring amino acid residue, for example, compared with the naturally occurring ApoC-II amino acid sequence (such as SEQ ID NOs: 13 or 14). In some examples, residues known to be important for LPL activation are not substituted, such as amino acid positions 17, 20, 23, and 24 of SEQ ID NO: 13 or corresponding positions in the peptides disclosed herein.

In some embodiments, the modification involves the substitution of one or more amino acids for amino acids having similar physiochemical and/or structural properties (e.g., “conservative” substitutions). Examples of conservative substitutions are shown below.

| Original Residue | Conservative Substitutions |
|---|---|
| Ala | Ser |
| Arg | Lys |
| Asn | Gln, His |
| Asp | Glu |
| Cys | Ser |
| Gln | Asn |
| Glu | Asp |
| His | Asn; Gln |
| Ile | Leu, Val |
| Leu | Ile; Val |
| Lys | Arg; Gln; Glu |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Conservative substitutions generally maintain (a) the structure of the peptide backbone in the area of the substitution, for example, as a helical conformation, (b) the charge or hydrophobicity of the molecule at the target site, or (c) the bulk of the side chain.

In other embodiments, one or more substitutions may be non-conservative, for instance changes in which (a) a hydrophilic residue, for example, seryl or threonyl, is substituted for (or by) a hydrophobic residue, for example, leucyl, isoleucyl, phenylalanyl, valyl or alanyl; (b) a cysteine or proline is substituted for (or by) any other residue; (c) a residue having an electropositive side chain, for example, lysyl, arginyl, or histadyl, is substituted for (or by) an electronegative residue, for example, glutamyl or aspartyl; or (d) a residue having a bulky side chain, for example, phenylalanine, is substituted for (or by) one not having a side chain, for example, glycine.

In additional embodiments, one or more amino acids may be added, for example as a hinge or linker region between domains of the peptide. In some examples, a proline residue is added between two alpha helical domains. Examples of such peptides include SEQ ID NO: 6 and SEQ ID NO: 23.

In addition to naturally occurring genetically encoded amino acids, one or more amino acid residues in the disclosed peptides may be substituted with naturally occurring non-encoded amino acids and/or synthetic amino acids. In some embodiments, the substitution(s) may increase lipid binding, increase resistance to proteolysis, or a combination thereof. In some examples, such amino acids include acylation of lysine ε-amino groups; N-alkylation of arginine, histidine, or lysine; alkylation of glutamic or aspartic carboxylic acid groups; deamidation of glutamine or asparagine; β-alanine and other omega-amino acids, such as 3-aminopropionic acid, 2,3-diaminopropionic acid, 4-aminobutyric acid and the like; α-aminoisobutyric acid; ε-aminohexanoic acid; 8-aminovaleric acid; N-methylglycine or sarcosine; ornithine; citrulline; t-butylalanine; t-butylglycine; N-methylisoleucine; phenylglycine; cyclohexylalanine; norleucine; naphthylalanine; 4-chlorophenylalanine; 2-fluorophenylalanine; 3-fluorophenylalanine; 4-fluorophenylalanine; penicillamine; 1,2,3,4-tetrahydroisoquinoline-3-carboxylic acid; β-2-thienylalanine; methionine sulfoxide; homoarginine; N-acetyl lysine; alpha-methyl-lysine; 2,4-diaminobutyric acid; 2,3-diaminobutyric acid; p-aminophenylalanine; N-methyl valine; homocysteine; homophenylalanine; homoserine; hydroxyproline; homoproline; N-methylated amino acids; peptoids (where side groups are appended to the nitrogen atom of the peptide backbone, rather than to the α-carbons); and β-peptides (where the amino group is bonded to the β carbon rather than the α-carbon).

While in certain embodiments, the amino acids of the disclosed ApoC-II mimetic peptides are L-amino acids, in other embodiments, one or more L-amino acids (such as 1, 2, 3, 4, or more amino acids) are replaced with an identical D-amino acid. In some examples, L-Lys is changed to D-Lys or L-Glu is changed to D-Glu. In other examples, the ApoC-II mimetic peptide includes one or more (such as 1, 2, 3, 4, or more) D-amino acids. In some examples, at least 1, 2, 3, or 4 C-terminal amino acids are D-amino acids. Specific, non-limiting examples include the peptides of SEQ ID NOs: 10, 17-20, 22, and 27-43.

In further embodiments, the disclosed peptides may include an N-terminal modification, a C-terminal modification, or both. In some examples, the peptide includes N-terminal acylation, C-terminal amidation, or both. In other examples, the modification includes an N-terminal or C-terminal fatty acid, for example, to increase binding of the peptide to lipoproteins. In some examples, the fatty acid is a C6-C18 fatty acid (from hexanoic acid to stearic acid). In specific examples, the fatty acid is octanoic acid (also known as caprylic acid) or myristic acid.

In other examples, N-terminal modifications include the desamino, N-lower alkyl, N-di-lower alkyl, constrained alkyls (e.g. branched, cyclic, fused, adamantyl), and N-acyl modifications. Exemplary C-terminal modifications include the amide, lower alkyl amide, constrained alkyls (e.g. branched, cyclic, fused, adamantyl) alkyl, dialkyl amide, and lower alkyl ester modifications. Lower alkyl is C1-C4 alkyl. In one non-limiting example, the peptide includes C-terminal amidation.

IgG and albumin have exceptionally long plasma half-lives, whereas most other human proteins exhibit rapid blood clearance. This long half-life of IgG and albumin is a result of interaction with neonatal Fc receptor (FcRn), which creates an intracellular protein reservoir that is protected from lysosomal degradation and subsequently recycled to the extracellular space. Genetic fusion between a therapeutic protein and either the Fc domain of IgG, albumin, or long, flexible polypeptide extensions has been introduced to extend protein circulation. FcRn can bind IgG at pH 6 but not bind at physiological pH (7.4), and this pH dependence is likely key to the mechanism by which FcRn extends IgG half-lives. It is thought that after uptake of IgG into cells, FcRn can bind to IgG in acidic endosomes, thereby avoiding degradation in the lysosome. IgG molecules are then returned to the cell surface by exocytosis and released back into circulation because FcRn has minimal affinity for IgG at extracellular pH 7.4.

In some examples, the ApoC-II mimetic peptides disclosed herein are linked to a human neonatal Fc receptor (FcRn) binding sequence, such as an IgG binding site (e.g., SEQ ID NO: 45), an albumin binding site (e.g., SEQ ID NO: 46), or a non-IgG/non-albumin FcRn binding site. See, e.g., U.S. Pat. Nos. 9,527,890; 9,012,603; 9,574,190; 10,046,023; 10,316,073; 10,588,935; Mezo et al., Proc. Natl. Acad. Sci. USA 105:2337-2342, 2008. These modified peptides can also include any of the modifications discussed above. In particular examples, the additional modifications include one or more of N-terminal acylation, C-terminal amidation, N-terminal fatty acid modification (such as octanoic acid or myristic acid), and C-terminal fatty acid modification (such as octanoic acid or myristic acid).

In some embodiments, the disclosed ApoC-II mimetic peptides include an amino acid sequence with at least 90% sequence identity (such as at least 90%, at least 91%, at least 92%, at least 93%, at least 94%, at least 95%, at least 96%, at least 97%, at least 98%, or at least 99% sequence identity) to the amino acid sequence of any one of SEQ ID NOs: 1-11 and 16-43. In other examples, the amino acid sequence of the ApoC-II mimetic peptides include or consist of the amino acid sequence of any one of SEQ ID NOs: 1-11 and 16-43. Exemplary hydrocarbon stapled or stitched ApoC-II mimetic peptides are provided in Table 1.

TABLE 1
Exemplary Apo-CII mimetic peptides

| | | SEQ |
|---|---|---|
| | | ID |
| Peptide | Sequence | NO: |
| CS-new | EK(R8)RDLYSK(S5)TAAMSTYTGIFTDQVLSVLKGEE | 1 |
| | R8-S5 stapled | |
| H2S | EKLRDLYSKSTAA(S5)STY(S5)GIFTDQVLSVLKGEE | 2 |
| | S5-S5 stapled | |
| H2LSL | EKLRDLYSKS(R8)AAMSTY(S5)GIFTDQVLSVLKGEE | 3 |
| | R8-S5 stapled | |
| H2LSR | EKLRDLYSKSTAAM(R8)TYTGIF(S5)DQVLSVLKGEE | 4 |
| | R8-S5 stapled | |
| SPH2S | STAAM(R8)TYTGIF(S5)DQVLSVLKGEE | 5 |
| | R8-S5 stapled | |
| HIS-new | EK(R8)RDLYSK(S5)TAPAMSTYTGIFTDQVLSVLKGEE | 6 |
| | R8-S5 stapled | |
| SSP-T1 | AM(R8)TYTGIF(S5)DQVLSVLKGEE | 7 |
| | R8-S5 stapled | |
| SSP-T2 | (R8)TYTGIF(S5)DQVLSVLKGEE | 8 |
| | R8-S5 stapled | |
| SSP-CTB | STAAM(R8)TYTGIF(S5)TDQVLSVLKGEE-amid | 9 |
| | R8-S5 stapled | |
| SSP-dE | STAAM(R8)TYTGIF(S5)TDQVLSVLKGee | 10 |
| | R8-S5 stapled | |
| SSP-SS | STAA(R8)STYTGI(S5)TDQVLSVLKGEE | 11 |
| | R8-S5 stapled | |
| CP | EKLRDLYSKSTAPAMSTYTGIFTDQVLSVLKGEE | 12 |
| ApoC-II | EKLRDLYSKSTAAMSTYTGIFTDQVLSVLKGEE | 13 |
| ApoC-II | AMSTYTGIFTDQVLSVLKGEE | 14 |
| truncated | | |
| D6PV | DYLKEVFEKLRDLYEKFTPAVSTYTGIFTDQVLSVLK | 15 |
| | GEE | |
| SPLL | AM(R8)TYTGIF(B5)DQVLSV(S8)KGEE-COOH | 16 |
| | R8-B5-S8 stitched | |
| SPLLee | A(Nle)(R8)TYTGIF(B5)DQVLSV(S8)KGee-COOH | 17 |
| | R8-B5-S8 stitched | |
| SPLLee-Sarc | A(Nle)(R8)TYTGIF(B5)DQVLSV(S8)K(Sarcosine)ee- | 18 |
| | COOH | |
| | R8-B5-S8 stitched | |
| SPLSee | A(Nle)(R8)TYTGIF(B5)DQV(S5)SVLKGee | 19 |
| | R8-B5-S5 stitched | |
| SPLSee-amK | A(Nle)(R8)TYTGIF(B5)DQV(S5)SVL(amK)Gee | 20 |
| | R8-B5-S5 stitched | |
| SPH2S-lactam | AM(R8)TYTGIF(S5)DQVLSVLCyclo[(K+)GE(E+)]- | 21 |
| | R8-S5 stapled | |
| 2SPee | A(Nle)(R8)TYTGIF(S5)DQ(R5)LSV(R5)KGee | 22 |
| | two staples R8-S5 and R5-R5 | |
| H2SP | EKLRDLYSKSTAPA(S5)STY(S5)GIFTDQVLSVLKGEE | 23 |
| | S5-S5 stapled | |
| H2CTS | EKLRDLYSKSTAAMSTYTGIFTDQ(R8)LSVLKG(S5)E | 24 |
| | R8-S5 stapled | |
| H1SH2F6064 | EK(R8)RDLYSK(S5)TAAFSTYFGIFTDQVLSVLKGEE | 25 |
| | R8-S5 stapled | |
| H1SH2F6065 | EK(R8)RDLYSK(S5)TAAFSTYTFIFTDQVLSVLKGEE | 26 |
| | R8-S5 stapled | |
| SPLSee/R8B5R5 | A(Nle)(R8)TYTGIF(B5)DQV(R5)SVLKGee | 27 |
| | R8-B5-R5 stitched | |
| SPLSee/S8B5S5 | A(Nle)(S8)TYTGIF(B5)DQV(S5)SVLKGee | 28 |
| | S8-B5-S5 stitched | |
| SPLSee- | A(Nle)(R8)TYTGIF(B5)DQV(R5)SVL(amK)Gee | 29 |
| amK/R8B5R5 | R8-B5-R5 stitched | |
| SPLSee- | A(Nle)(S8)TYTGIF(B5)DQV(S5)SVL(amK)Gee | 30 |
| amK/S8B5S5 | S8-B5-S5 stitched | |
| SPLLee-Sarc-19 | (R8)TYTGIF(B5)DQVLSV(S8)K(Sarcosine)ee | 31 |
| | R8-B5-S8 stitched | |
| SPLLee-Sarc- | a(nle)(R8)TYTGIF(B5)DQVLSV(S8)K(Sarcosine)ee | 32 |
| Da | R8-B5-S8 stitched | |
| SPLLee-Sarc- | a(nle)(R8)TYTGIF(B5)DQVLSV(S8)k(Sarcosine)ee | 33 |
| Dak | R8-B5-S8 stitched | |
| SPH2S-Sarc | Ac-A(Nle)(R8)TYTGIF(S5)DQVLSVLK(Sarcosine)ee | 34 |
| | R8-S5 stapled, N-terminus acetylated | |
| SPH2S-Sarc-Da | a(nle)(R8)TYTGIF(S5)DQVLSVLK(Sarcosine)ee | 35 |
| | R8-S5 stapled | |
| SPH2S-Sarc- | a(nle)(R8)TYTGIF(S5)DQVLSVLk(Sarcosine)ee | 36 |
| Dak | R8-S5 stapled | |
| Octa- SPH2S- | Octanoic - | 37 |
| Sarc | A(Nle)(R8)TYTGIF(S5)DQVLSVLK(Sarcosine)ee | |
| | R8-S5 stapled | |
| Octa- SPH2S- | Octanoic - | 38 |
| Sarc-4e | ANle(R8)TYTGIF(S5)DQVLSVLK(Sarcosine)eeee | |
| | R8-S5 stapled | |
| Octa- SPH2S- | Octanoic-(R8)TYTGIF(S5)DQVLSVLK(Sarcosine)eeee | 39 |
| Sarc-19-4e | R8-S5 stapled | |
| Ac-SPH2S- | Ac-ANle(R8)TYTGIF(S5)DQVLSVLK(Sarcosine)eeee | 40 |
| Sarc-4e | R8-S5 stapled | |
| Octa-SPLLee- | Octanoic- | 41 |
| Sarc | A(Nle)(R8)TYTGIF(B5)DQVLSV(S8)K(Sarcosine)ee | |
| | R8-B5-S8 stitched | |
| Octa-SPLLee- | Octanoic-(R8)TYTGIF(B5)DQVLSV(S8)K(Sarcosine)ee | 42 |
| Sarc-19 | R8-B5-S8 stitched | |
| Octa-SPLLee- | Octanoic- | 43 |
| Sarc-Aib | Aib(Nle)(R8)TYTGIF(B5)DQVLSV(S8)K(Sarcosine)ee | |
| | R8-B5-S8 stitched | |

(S5) = (S)-α-methyl, α-pentenylglycine
(S8) = (S)-α-methyl, α-octenylglycine
(B5) = bis-pentenylglycine
(R5) = (R)-α-methyl, α-pentenylglycine
(R8) = (R)-α-methyl, α-octenylglycine
Nle = Norleucine
nle = D-isomer of Norleucine
Sarcosine = N-methylglycine
amK = alpha-methyl-Lysine
(K+) and (E+) = Lys and Glu connected by lactam bridge
e = D-isomer of Glu
k = D-isomer of Lys
a = D-isomer of Ala
Aib = 2-aminoisobutyric acid

In some embodiments, the disclosed ApoC-II mimetic peptides can be isolated from various sources. For example, a disclosed peptide can be produced by peptide synthesis (such as solid-phase or solution-phase synthesis), followed by reaction under appropriate conditions to create the one or more covalent linkages between two or more non-contiguous amino acids of the peptides. In some examples, the covalent linkage is a hydrocarbon staple or hydrocarbon stitch and the hydrocarbon staple or hydrocarbon stitch is produced by ruthenium-catalyzed metathesis or bis ring-closing metathesis.

In some embodiments, the ApoC-II mimetic peptides disclosed herein retain one or more activities including increasing LPL activity, decreasing ApoC-III binding to VLDL, reducing serum triglyceride levels in a subject, and reducing total serum cholesterol levels in a subject. In some examples, the disclosed peptides increase LPL activity by at least about 2-10-fold compared to a control (such as a peptide with native ApoC-II (59-79) sequence or with no activator) in an LPL activation assay. In other examples, the disclosed peptides decrease binding of ApoC-III to VLDL by at least about 10% compared to a control (such as no peptide/buffer) in an ApoC-III displacement assay, for example as described in Wolska et al. (Sci. Transl. Med. 12, eaaw7905, 2020). In further examples, the disclosed peptides decrease serum triglycerides by at least about 25% in one hour compared to a control (e.g., no peptide/buffer) when injected IP in ApoC-II-KO mice. In additional examples, the disclosed peptides decrease serum cholesterol by at least about 25% compared to a control (e.g., no peptide/buffer) when injected IP in ApoC-II-KO mice.

### III. Pharmaceutical Compositions and Methods of Use

In exemplary applications, compositions including one or more of the disclosed ApoC-II mimetic peptides disclosed herein are administered to a subject suffering from a dyslipidemic disorder, such as hypertriglyceridemia or hypercholesterolemia in a therapeutically effective amount. In other examples, ApoC-II mimetic peptides disclosed herein are administered to a subject with a genetic deficiency of apoC-II, other forms of hypertriglyceridemia from LPL deficiency, and/or conditions characterized by excess apoC-III. Amounts effective for this use will depend upon the severity of the disorder and the general state of the subject's health. A therapeutically effective amount of the compound is that which provides either subjective relief of one or more symptoms or an objectively identifiable improvement as noted by a clinician or other qualified observer.

An ApoC-II mimetic peptide can be administered by any means known to one of skill in the art (see, e.g., Banga, “Parenteral Controlled Delivery of Therapeutic Peptides and Proteins,” in Therapeutic Peptides and Proteins, Technomic Publishing Co., Inc., Lancaster, P A, 1995), such as by intramuscular, subcutaneous, or intravenous injection, or oral, nasal or inhalation, or anal administration. In one embodiment, administration is by subcutaneous injection. In another embodiment, administration is oral. To extend the time during which the ApoC-II mimetic peptide is available to inhibit or treat a dyslipidemic disorder, the peptide can be provided as an implant, an oily injection, or as a particulate system. The particulate system can be a microparticle, a microcapsule, a microsphere, a nanocapsule, or similar particle. In specific embodiments, the ApoC-II mimetic peptide that is administered includes any one of SEQ ID NOs: 1-11 and 16-43.

In some examples, the methods include administering a therapeutically effective amount of a disclosed ApoC-II mimetic peptide to a subject in need thereof. In some examples, the subject is a human or veterinary subject. In particular examples, the subject is human.

In some examples, the subject has hypertriglyceridemia. Normal or desirable serum triglycerides in a human subject is less than 150 mg/dL. The borderline high triglyceride range is 150-199 mg/dL, high triglyceride range is 200-499 mg/dL, and very high triglyceride range is 500 mg/dL or more. Thus, in some examples, the subject being treated has serum triglycerides of 150 mg/dL or more. The subject is administered a therapeutically effective amount of one or more of the disclosed peptides, such as an amount that results in a decrease in serum triglyceride levels by about at least 5% (such as at least about 5%, at least about 10%, at least about 15%, at least about 20%, at least about 25%, at least about 30%, at least about 40%, at least about 50% or more), for example, compared to triglyceride levels prior to administration of the peptide. In other examples, a therapeutically effective amount of the disclosed peptides reduces serum triglycerides in the subject to less than 150 mg/dL.

In some examples, the subject has hypercholesterolemia. Normal or desirable total cholesterol in an adult human subject is less than 200 mg/dL (less than 170 mg/dL for children). The borderline high total cholesterol range for adults is 200-239 mg/dL, and the high total cholesterol range is 240 mg/dL or more (170-199 mg/dL and 200 mg/dL or more, respectively, in children). Thus, in some examples, the subject being treated is an adult and has a serum total cholesterol level of 200 mg/dL or more. The subject is administered a therapeutically effective amount of one or more of the disclosed peptides, such as an amount that results in a decrease in serum total cholesterol levels by about at least 5% (such as at least about 5%, at least about 10%, at least about 15%, at least about 20%, at least about 25%, at least about 30%, at least about 40%, at least about 50% or more), for example, compared to total cholesterol levels prior to administration of the peptide. In other examples, a therapeutically effective amount of the disclosed peptides reduces serum total cholesterol in an adult subject to less than 200 mg/dL.

One or more than one of the provided ApoC-II mimetic peptides can be combined with one or more pharmaceutically acceptable carriers or excipients for administration to human or animal subjects. Remington: The Science and Practice of Pharmacy, 22^{nd} ed., London, UK: Pharmaceutical Press (2013), describes compositions and formulations suitable for pharmaceutical delivery of one or more therapeutic compounds or molecules, such as one or more peptides alone or in combination with additional pharmaceutical agents. Examples of suitable pharmaceutically acceptable carriers, vehicles, or excipients include sterile aqueous or non-aqueous solutions, suspensions, and/or emulsions. Examples of non-aqueous solvents include propylene glycol, polyethylene glycol, vegetable oils such as olive oil, and injectable organic esters such as ethyl oleate. Aqueous carriers include water, alcoholic/aqueous solutions, emulsions, or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Additional pharmaceutically acceptable carriers include carbohydrates (for example, glucose, sucrose, or dextrans), antioxidants (such as ascorbic acid or glutathione), chelating agents, low molecular weight proteins, lipids, wetting agents, emulsifying agents, dispersing agents, and preservatives.

In general, the formulations are prepared by uniformly and intimately bringing into association one or more of the disclosed peptides with the pharmaceutically acceptable carrier(s) or excipient(s). Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze-dried (lyophilized) condition requiring only the addition of a sterile liquid carrier, for example, water or saline for injections, immediately prior to use. Extemporaneous injection solutions and suspensions may be prepared from sterile powders, granules and tablets commonly used by one of ordinary skill in the art.

The peptides and pharmaceutical compositions provided herein, including those for use in treating dyslipidemic disorders, may be administered through different routes, such as oral, including buccal and sublingual, rectal, parenteral, aerosol, nasal, intravenous, intramuscular, subcutaneous, intradermal, and topical. They may be administered in different forms, including but not limited to solutions, emulsions and suspensions, microspheres, particles, microparticles, nanoparticles, and liposomes.

The amount of the peptide or pharmaceutical composition that will be effective depends on the nature of the disorder or condition to be treated, as well as the stage of the disorder or condition. Effective amounts can be determined by standard clinical techniques. The precise dose to be employed in the formulation will also depend on the route of administration, and should be decided according to the judgment of the health care practitioner and each subject's circumstances. An example of such a dosage range is 0.1 to 200 mg/kg body weight (for example, 0.1 to 10 mg/kg, 1-25 mg/kg, 5-50 mg/kg, 25-75 mg/kg, 50-100 mg/kg, 75-150 mg/kg, or 100-200 mg/kg) in single or divided doses. Another example of a dosage range is 1.0 to 100 mg/kg body weight in single or divided doses. In some embodiments, the disclosed peptides or pharmaceutical compositions are administered twice per day, once daily, every other day, weekly, or less frequently. In one specific example, the subject is administered the peptide or composition subcutaneously once weekly. In another specific example, the subject is administered the peptide or composition orally once per day. Treatment may continue indefinitely, or may be discontinued when a target clinical parameter is reached, such as normal range triglyceride or total cholesterol levels.

The specific dose level and frequency of dosage for any particular subject may be varied and will depend upon a variety of factors, including the activity of the specific compound, the metabolic stability and length of action of that compound, the age, body weight, general health, sex, diet, mode and time of administration, rate of excretion, drug combination, and severity of the condition of the subject undergoing therapy.

The disclosed peptides or pharmaceutical compositions can be administered at about the same dose throughout a treatment period, in an escalating dose regimen, or in a loading-dose regime (e.g., in which the loading dose is about two to five times the maintenance dose). In some embodiments, the dose is varied during the course of a treatment based on the condition of the subject being treated, the severity of the disease or condition, the apparent response to the therapy, and/or other factors as judged by one of ordinary skill in the art.

### EXAMPLES

The following examples are provided to illustrate certain particular features and/or embodiments. These examples should not be construed to limit the disclosure to the particular features or embodiments described.

### Example 1

### Production of Peptides and Evaluation of Activity In Vitro

LPL activity was determined by measuring the generation of NEFA. In a 96-well plate, 0-10 μM of an apoC-II mimetic peptide, 7.45 nM of LPL from bovine milk (Sigma-Aldrich), apoC-II deficient ethylenediaminetetraacetic acid (EDTA)-plasma (diluted with PBS to a final TG concentration of 3 mg/dL), 10 IU/ml heparin, 1% bovine serum albumin (BSA) (ICN Biomedicals), and PBS (Life Technologies) were combined in a final reaction volume of 50 μL, pre-incubated on ice for 30 minutes, and incubated at 37° C. for 1 hour. NEFA were measured with a coupled enzyme reaction (FUJIFILM Wako Diagnostics), in a SpectraMax Plus 384 Microplate Reader.

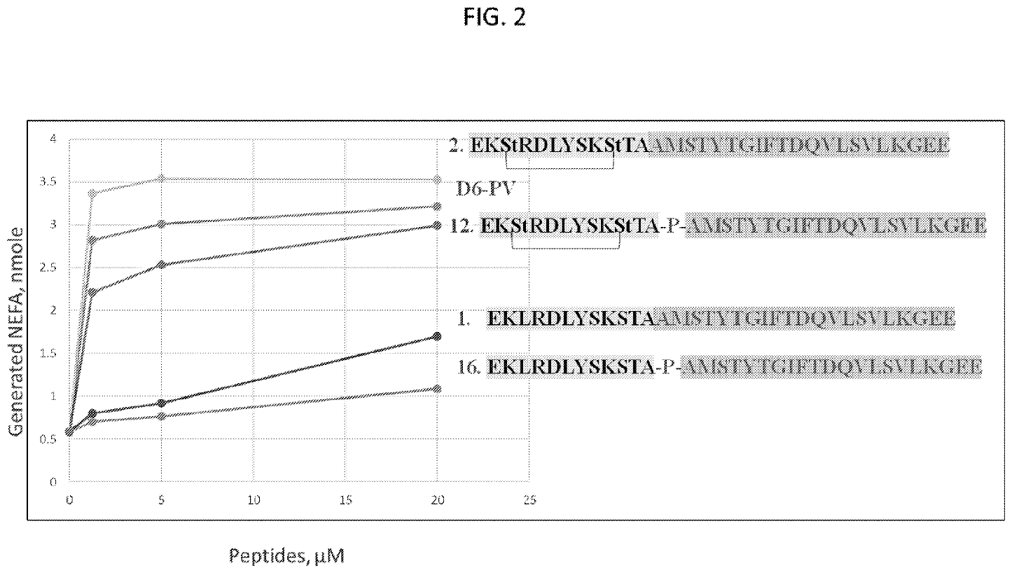

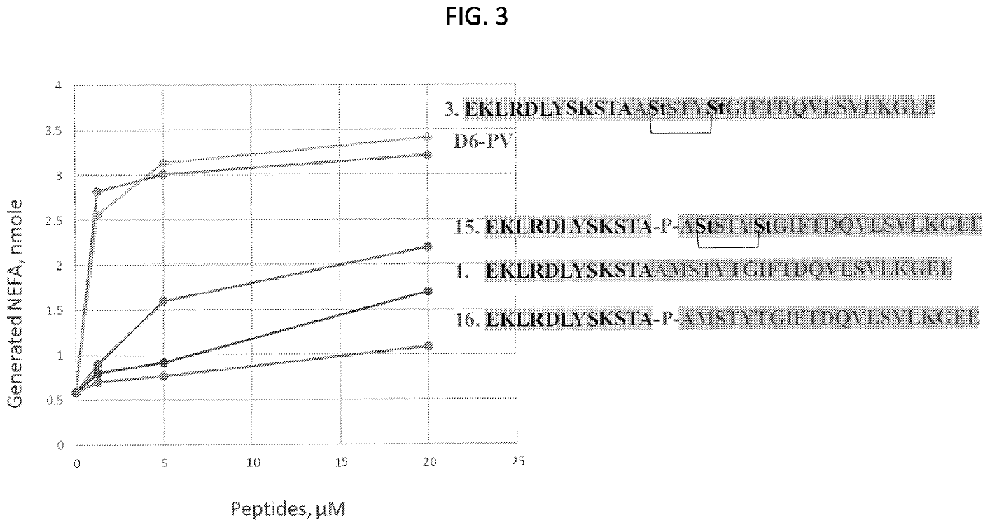

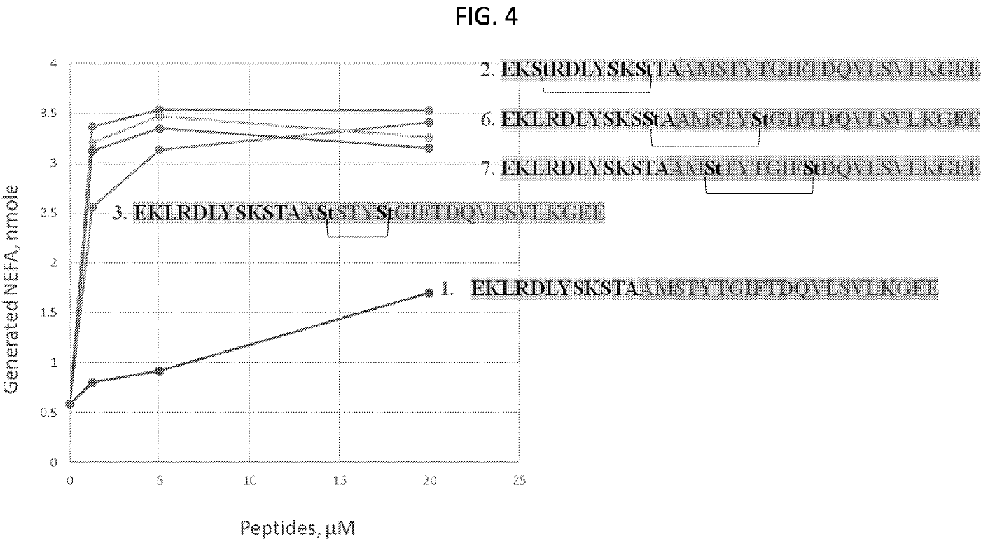

The effect of peptide length, sequence, and length and position of the staple was evaluated. In initial experiments, the effect of introducing a proline between two helixes in combination with a long or short hydrocarbon staple was tested. As shown in FIGS. 2 and 3, introducing a proline between two helixes reduced the peptide's efficacy. Short and long staples were effective (FIGS. 3 and 4).

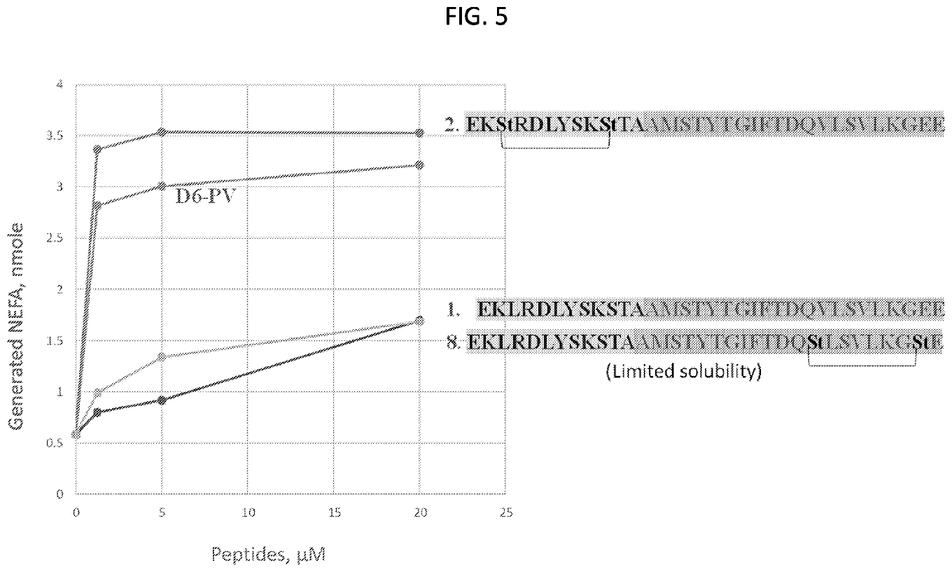

The effect of position of the staple (N-terminal vs. C-terminal) was also tested. Placing the staple near the C-terminus of the peptide reduced the efficacy and solubility of the peptide compared to the same peptide with the staple near the N-terminus (FIG. 5).

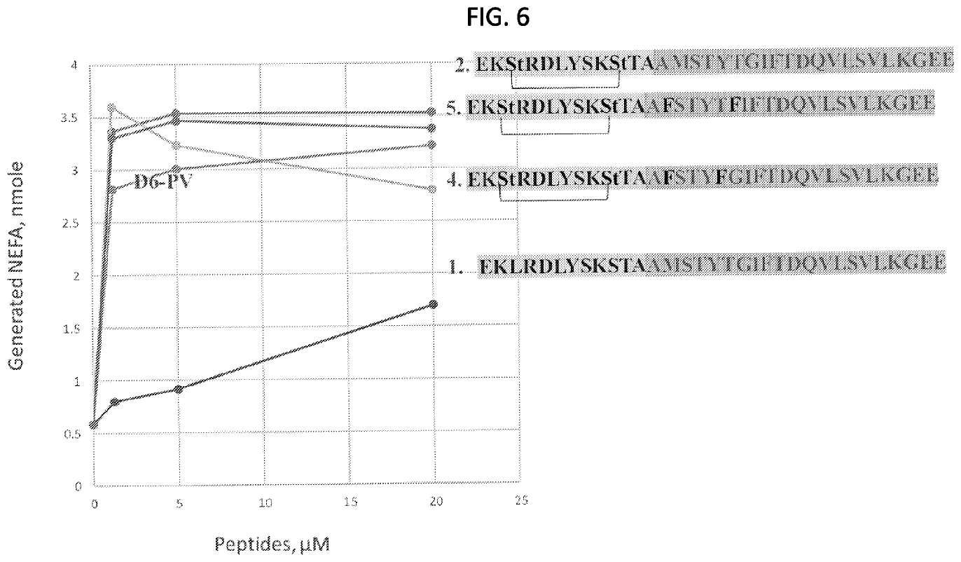

Peptides with phenylalanine substitutions at positions 60 and 64 (SEQ ID NO: 25) or 60 and 65 (SEQ ID NO: 26) were tested. The substitutions at positions 60 and 65 did not affect peptide efficacy for activation of LPL; however substitutions at positions 60 and 64 did reduce the efficacy (FIG. 6).

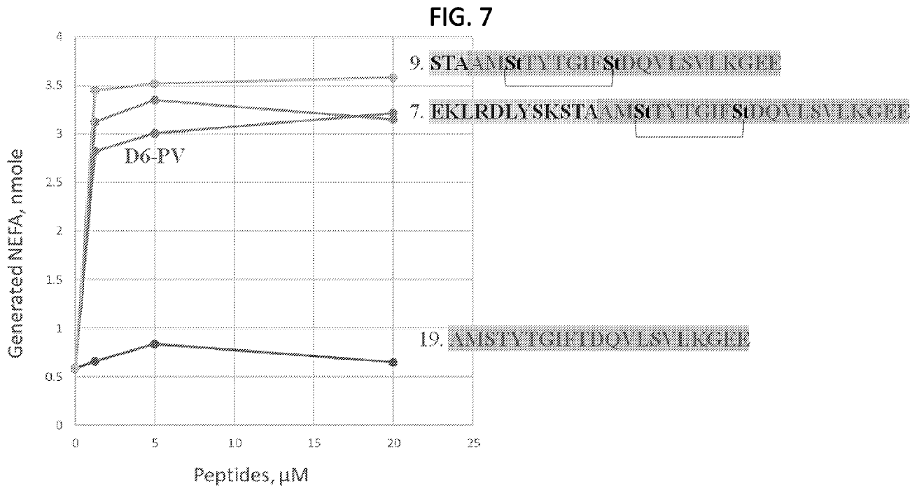

Truncation of the peptide to S56 (SEQ ID NO: 5) increased efficacy compared to a peptide with the staple in the same position, but starting at E47 (SEQ ID NO: 4), as shown in FIG. 7.

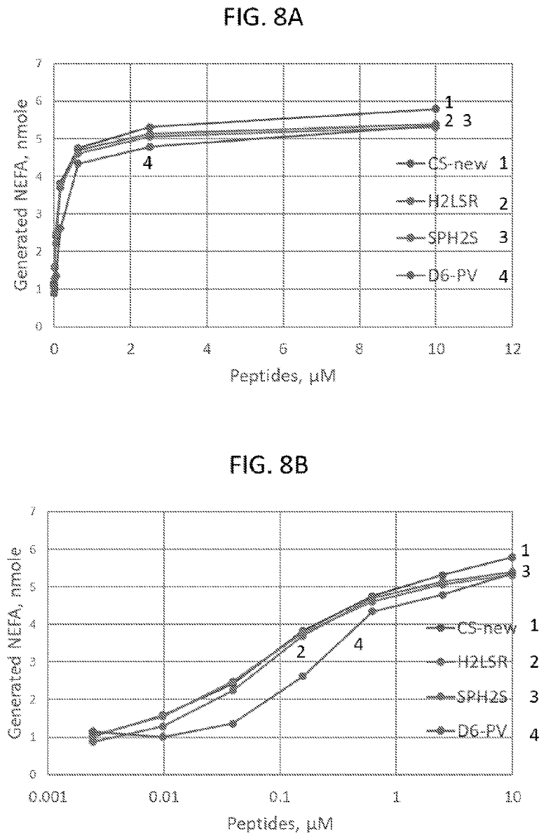

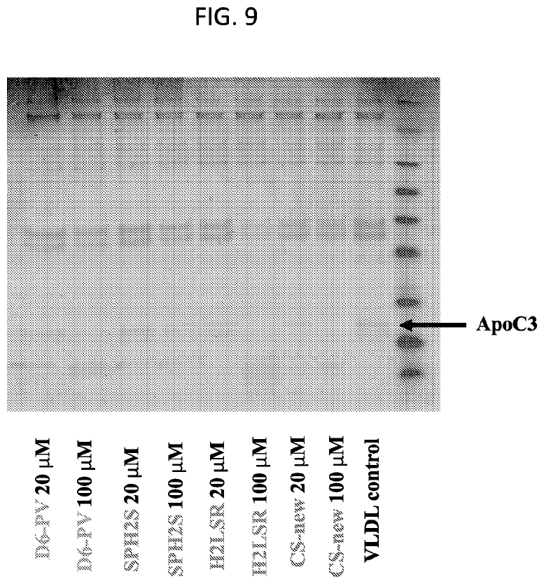

Selected peptides were tested for activation of LPL in over extended concentrations (FIGS. 8A and 8B). As seen when the data were analyzed on a logarithmic scale (FIG. 8B), the stapled peptides were more effective at activating LPL than the control D6-PV peptide. The peptides also displaced ApoC-III from VLDL (FIG. 9) similarly to D6-PV removing ApoC-III from triglyceride-rich lipoproteins. Therefore, these peptides also might cause ApoC-III clearance by the kidney and remnant particles depleted of ApoC-III to be cleared by the liver

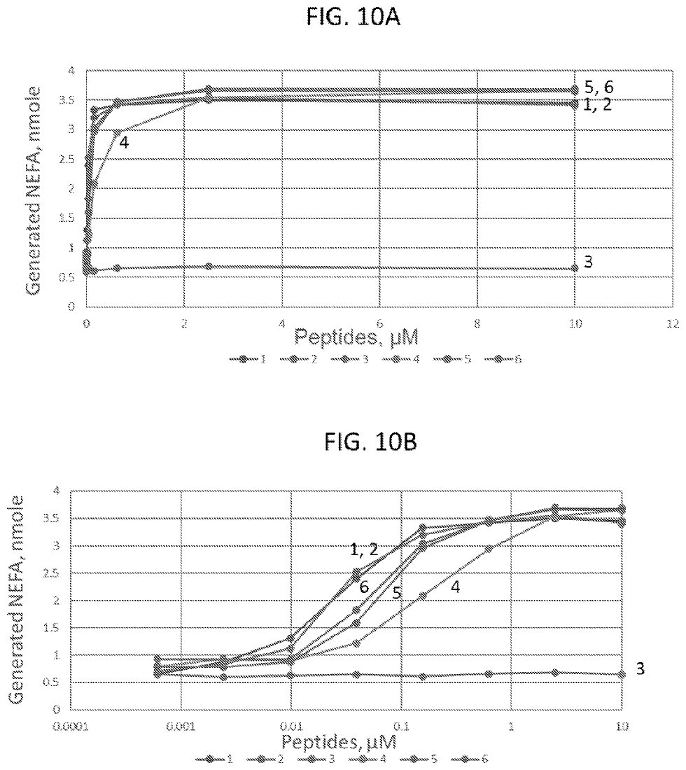

Additional peptides with further modifications were also tested for LPL activation. As seen in FIGS. 10A and 10B, the modifications increased efficacy compared to the initial truncated peptide (SEQ ID NO: 5). In this experiment peptides were matched by molar concentration. In this case smaller peptides (such as SPH2S) are less concentrated in terms of mass (mg/ml) concentration. Thus, it appears that displacement is of ApoC-III may be mass dependent.

### Example 2

### Evaluation of Peptide Activity In Vivo

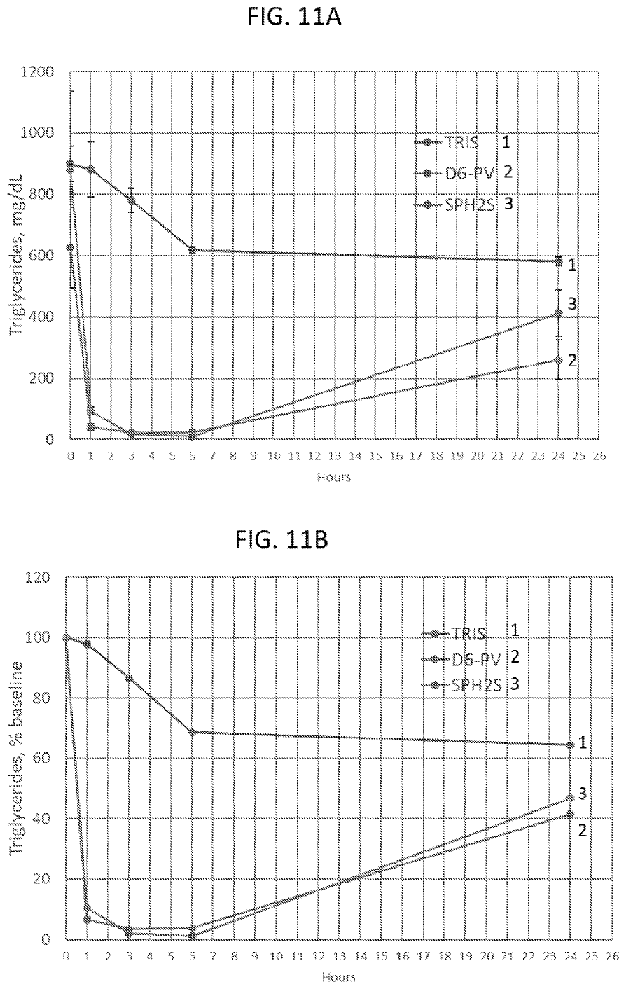

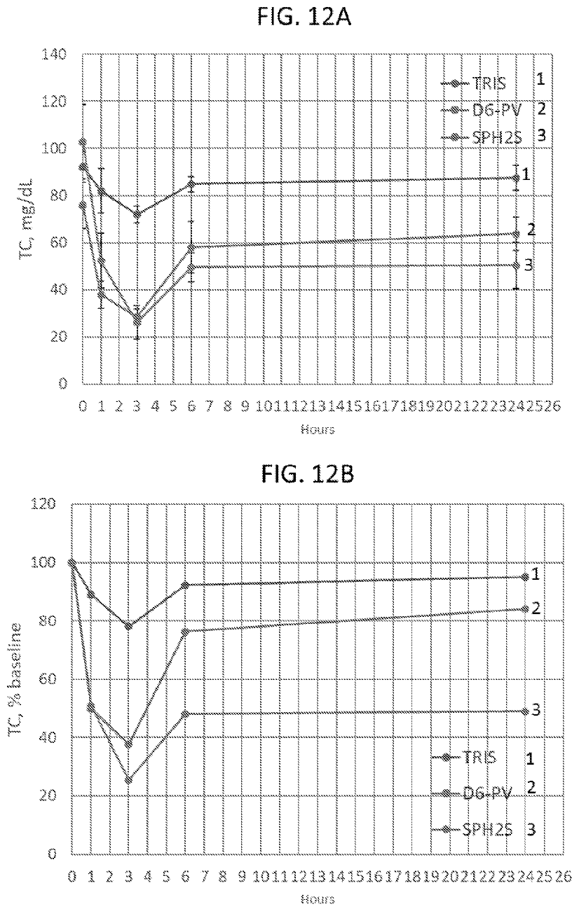

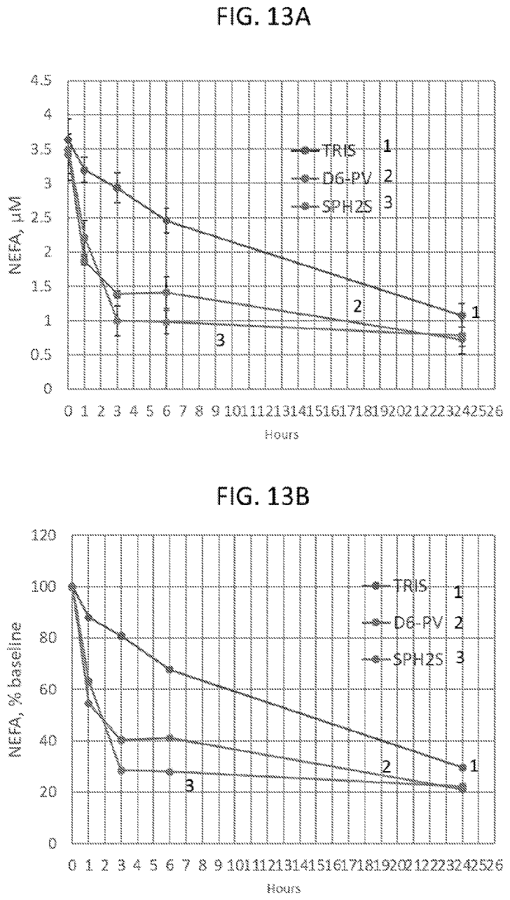

A study was performed comparing the effect of D6-PV (SEQ ID NO: 15) and SPH2S (SEQ ID NO: 5) peptides in apoC-II knockout mice. Mice were given a single intraperitoneal injection of the peptide (5 μmole/kg) and blood was collected at 1 hour, 3 hours, 6 hours and 24 hours after injection. Triglycerides, total cholesterol, and non-esterified fatty acids (NEFA) were measured. Both D6-PV and SPH2S decreased triglycerides to a similar extent (FIGS. 11A and 11B) compared to control. SPH2S was more effective than D6-PV at decreasing total cholesterol (FIGS. 12A and 12B) and NEFA (FIGS. 13A and 13B).

### Example 3

### Further Peptide Evaluation

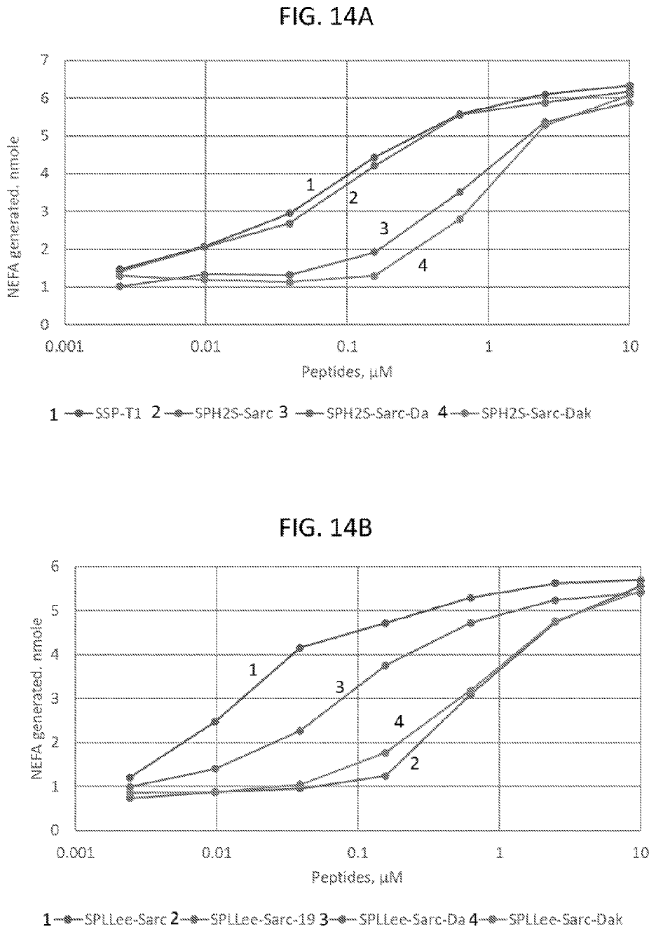

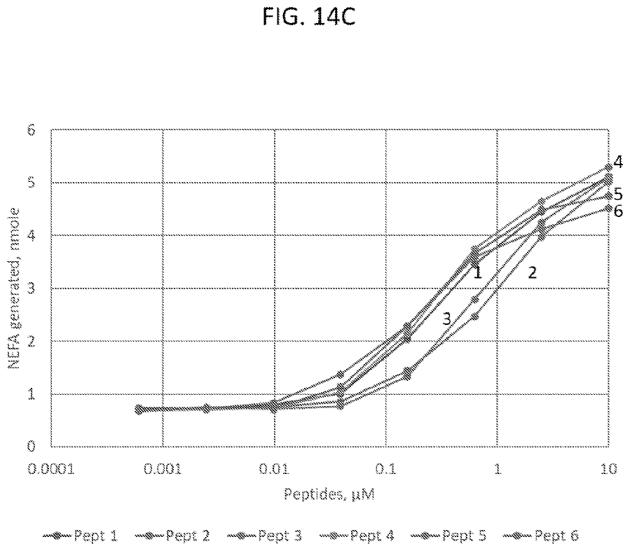

LPL activation activity of additional peptides was evaluated in vitro as described in Example 1. The results are shown in FIGS. 14A-14C. Substitution of G77 in peptide SEQ ID NO: 7 to Sarcosine (SEQ ID NO: 34) did not affect LPL activation, but substituting A59 and Nle60 (SEQ ID NO: 35) and A59, Nle60 and K76 with corresponding D-isomers (SEQ ID NO: 36) decreased LPL activation abilities of these stapled peptides (FIG. 14A). This was also true for stitched peptides tested (FIG. 14B). Addition of octanoic acid to the N-terminus of SEQ ID NO: 34 did not affect LPL activation (SEQ ID NO: 37), but truncating it to R8 (SEQ ID NO: 39) decreased peptide activity (FIG. 14C).

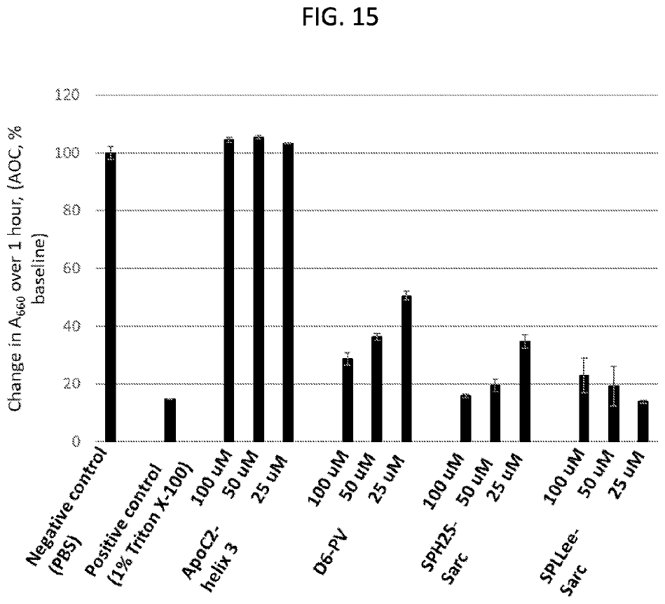

Tested peptides were able to solubilize DPMC vesicles (FIG. 15). In contrast, the ApoC2-helix 3 peptide did not. PBS as a negative control or 1% Triton as a positive control or 0.5 mM peptides were added to homogenous DMPC (Avanti 850345C) vesicles (0.5 mg/mL) in PBS, pH 7.4. In vitro turbidity change was monitored by following the absorbance at 660 nm for 1 hour at 24° C. Absorbance over time was plotted and the areas under the curve (AOC) were calculated.

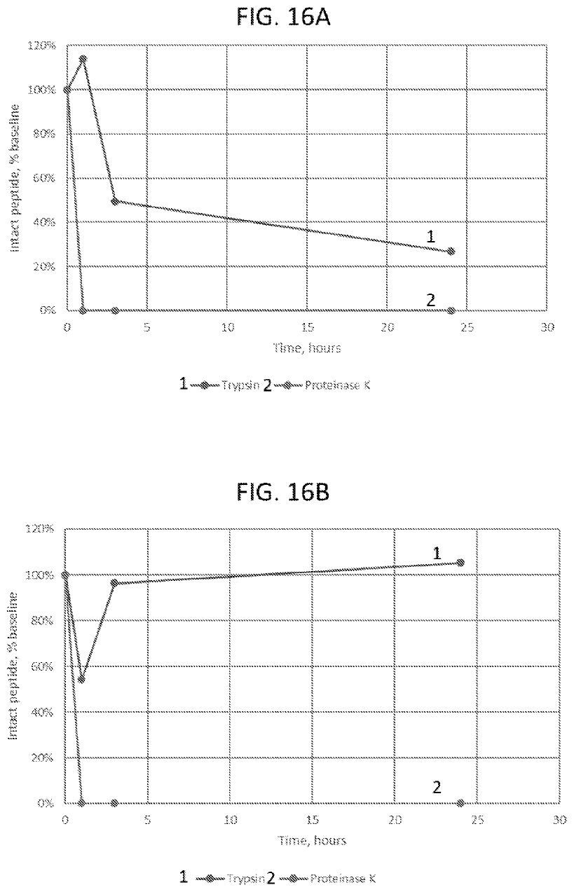

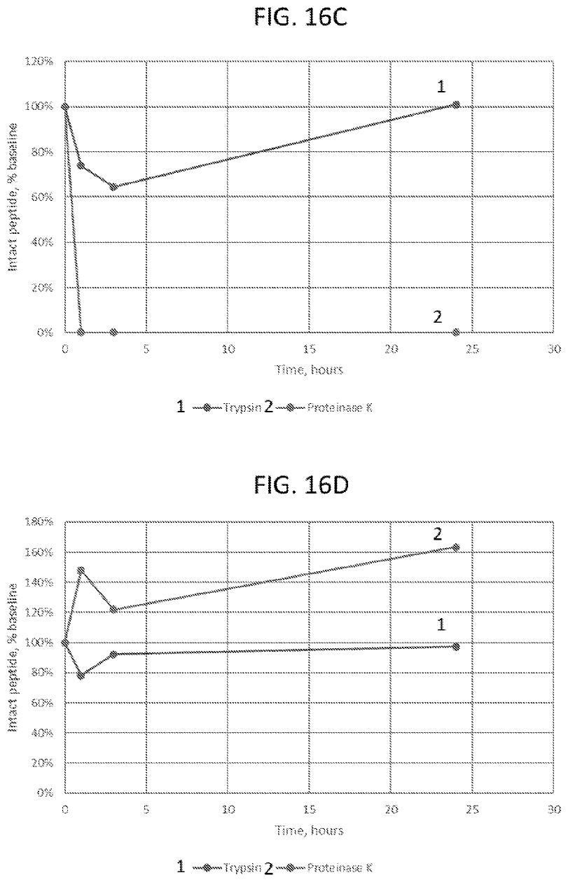

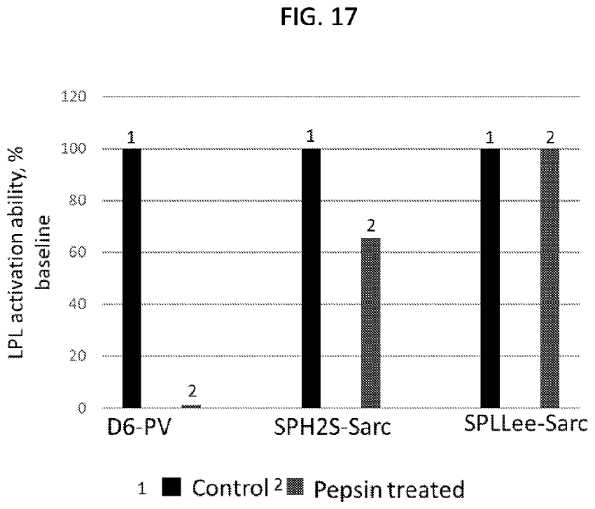

Peptide resistance to proteolysis was also tested. Peptides at 0.1 mM concentration were incubated at 37° C. in 20 mM ammonium bicarbonate buffer at pH 8.2 with 10 μg/ml trypsin or 16 μg/kg Proteinase K. The amount of intact peptide was monitored over 24-hour time period by MALDI-TOF using internal standards (Bombesin, ACTH (1-17), ACTH (18-39), and Insulin) to quantify the relative amount of peptide in each sample. As shown in FIGS. 16A-16D, the tested peptides had varying degrees of resistance to trypsin and proteinase K. In addition, peptides were tested for LPL activation ability following pepsin treatment. Peptides (1 mM stock) were prepared in 5 mM TRIS, pH 8.2. Then 50 μL of the stock was added to 450 μl of 20 mM Acetic Acid buffer, pH 1.92 with or without 1 mg/ml Pepsin A (Sigma Aldrich P6887-1G). After 1 hour incubation at 37° C. with 800 min-1 shaking, aliquots were taken and mixed with 100 mM TRIS, pH 7.4 to inhibit Pepsin A to a final concentration of 10 UM of peptides. These mixtures were used for LPL activation assays. The assays were carried out as described in Example 1. In vitro generated NEFA were measured by the colorimetric assay. SPH2S-Sarc and SPLLee-Sarc peptides showed resistance to pepsin treatment (FIG. 17).

In view of the many possible embodiments to which the principles of the disclosure may be applied, it should be recognized that the illustrated embodiments are only examples and should not be taken as limiting the scope of the invention. Rather, the scope of the invention is defined by the following claims. We therefore claim as our invention all that comes within the scope and spirit of these claims.

1. An isolated ApoC-II mimetic peptide of 19-35 amino acids comprising one or two helical domains with one or more covalent linkages joining at least two non-contiguous amino acids of the peptide, wherein at least one of the helical domains is an amphipathic helical domain.

2. The peptide of claim 1, wherein the covalent linkage joining the at least two non-contiguous amino acids is between two amino acids on the hydrophobic side of the amphipathic helical domain.

3. The peptide of claim 1, wherein the one or more covalent linkages is a hydrocarbon staple, a hydrocarbon stitch, a lactam bridge, or a disulfide bond.

4. The peptide of claim 3, wherein the hydrocarbon staple or hydrocarbon stitch comprises a linkage comprising one or more of (S)-α-methyl, α-pentenylglycine (S5), (S)-α-methyl, α-octenylglycine (S8), bis-pentenylglycine (B5), (R)-α-methyl, α-pentenylglycine (R5), and (R)-α-methyl, α-octenylglycine (R8).

5. The peptide of claim 1, wherein the peptide comprises one or more additional modifications.

6. The peptide of claim 5, wherein the additional modification comprises one or more amino acid substitutions, additions, or deletions; C-terminal amidation; N-terminal acylation, one or more D-isomer amino acids; a modified amino acid; an N-terminal fatty acid; a C-terminal fatty acid; a human neonatal Fc receptor (FcRn) binding sequence; or a combination of two or more thereof.

7. The peptide of claim 6, wherein the fatty acid is octanoic acid or myristic acid and/or wherein the human neonatal Fc receptor (FcRn) binding sequence comprises SEQ ID NO: 45 or SEQ ID NO: 46.

8. (canceled)

9. The peptide of claim 1, wherein the peptide:
comprises an amino acid sequence with at least 90% identity to the amino acid sequence of any one of SEQ ID NOs: 34, 17, 1-11, 16, 18-33, and 35-43;
comprises the amino acid sequence of any one of SEQ ID NOs: 34, 17, 1-11, 16, 18-33, and 35-43; or
consists of the amino acid sequence of any one of SEQ ID NOs: 34, 17, 1-11, 16, 18-33, and 35-43.

10-11. (canceled)

12. The peptide of claim 1, wherein the peptide is 19-25 amino acids long.

13. The peptide of claim 1, wherein the peptide activates lipoprotein lipase and/or wherein the peptide displaces ApoC-III from very low density lipoprotein.

14. (canceled)

15. A pharmaceutical composition comprising the peptide of claim 1 and a pharmaceutically acceptable carrier.

16. The pharmaceutical composition of claim 15, wherein the composition is formulated for intravenous administration, subcutaneous administration, or oral administration.

17. A method of decreasing triglyceride levels or cholesterol levels in a subject, comprising administering to the subject an effective amount of the peptide of claim 1.

18. The method of claim 17, wherein the subject has hypertriglyceridemia.

19. The method of claim 18, wherein the subject has lipoprotein lipase deficiency.

20. The method of claim 17, wherein the subject has a pre-treatment serum triglyceride level of the subject is 150 mg/dL or more.

21. (canceled)

22. The method of claim 17, wherein the subject has hypercholesterolemia.

23. The method of claim 17, wherein the administering comprises intravenous administration, subcutaneous injection, or oral administration.

24. (canceled)

25. A method of making the peptide of claim 1, comprising producing the peptide recombinantly.

26. The method of claim 25, wherein the peptide is produced by chemical synthesis.
